(19) United States
(12) Patent Application Publication
(10) Pub. No.: US 2025/0025457 A1
(43) Pub. Date: Jan. 23, 2025

(54) STABLE FORMULATIONS OF BUPRENORPHINE

(71) Applicant: Scott Hostetler, Westfield, IN (US)

(72) Inventor: Scott Hostetler, Westfield, IN (US)

(73) Assignee: Elanco US Inc., Greenfield, IN (US)

(21) Appl. No.: 18/694,487

(22) PCT Filed: Sep. 30, 2022
(86) PCT No.: PCT/US2022/045421

§ 371 Date: Mar. 22, 2024

Related U.S. Application Data
(60) Provisional application No. 63/250,723, filed on Sep. 30, 2021.
(60) Provisional application No. 63/348,962, filed on Jun. 3, 2022.

(51) Int. Cl. A61K 31/485; A61K 9/00; A61K 47/10; A61K 47/18

(52) U.S. Cl. CPC A61K 31/485; A61K 9/0017; A61K 47/10; A61K 47/18

(57) ABSTRACT

Methods of providing pain relief to animals, for example cats, by administering a formulation comprising buprenorphine or salt thereof are described. A formulation comprising buprenorphine or salt thereof and an additive selected from butylated hydroxyanisole (BHA), butylated hydroxytoluene (BHT) and combinations thereof and methods of storage and using such formulations are also provided.

### CROSS-REFERENCE TO RELATED APPLICATIONS

This patent application is an international patent application which claims priority to U.S. Provisional Application No. 63/250,723 filed on Sep. 30, 2021 and to U.S. Provisional Application No. 63/348,962 filed on Jun. 3, 2022, the disclosures of which is incorporated herein in its entirety.

### FIELD

The present disclosure relates to formulations, methods, uses and devices containing buprenorphine or salt thereof. This disclosure provides methods of providing pain relief to animals, for example cats. The present disclosure relates to stable formulations of buprenorphine or salt thereof. This disclosure further provides methods of storing and shipping stable formulations of buprenorphine or salt thereof and related methods of use for treating or controlling pain such as postoperative pain associated with surgical procedures in cats.

### BACKGROUND

Buprenorphine is a potent, partial agonist of the u-opioid receptor that has been shown to be effective to control pain in a wide range of patients when delivered by a number of different routes of administration, including transdermally, intravenously, intramuscularly, subcutaneously, epidurally, intrathecally, or sublingually.

ZORBIUM (Buprenorphine Transdermal Solution) is long-acting, transdermal formulation of buprenorphine useful, e.g., to control postoperative pain associated with surgical procedures in cats. The initial formulation for ZORBIUM required refrigerated storage (5° C.) to prevent degradation of the active ingredient (buprenorphine hydrochloride). Even with refrigerated storage, shelf-life is limited to only about 6 months. Cold chain storage and distribution of a pharmaceutical product is not only inconvenient and costly but can limit the range of distribution of the product to locations that have proper storage equipment and have sufficient storage space for the product. Thus, there exists a need for formulations containing buprenorphine that do not require refrigeration for distribution and storage. Further, there remains a need for stable formulations containing buprenorphine that exhibit an enhanced shelf life (e.g., 6 months or more) even under storage conditions at room temperature and/or elevated temperatures.

### SUMMARY OF THE INVENTION

One aspect of the invention is a formulation comprising buprenorphine or salt thereof, and at least one additive selected from the group comprising butylated hydroxyanisole (BHA), butylated hydroxytoluene (BHT) and combinations thereof.

Another aspect of the invention is a method for storing the formulation comprising storing the formulation at a temperature greater than 5° C.

Yet another aspect of the invention is a method for shipping the formulation comprising shipping the formulation at a temperature greater than 5° C.

A further aspect of the invention is a method for reducing pain in a mammal subject in need thereof comprising administering to the subject the formulation.

### BRIEF DESCRIPTION OF THE DRAWINGS

FIG. 1 shows UPLC (ultra-performance liquid chromatography) and MS (mass spectrometry) parameters.

FIG. 2 and FIG. 3 show UPLC chromatographs of a temperature stressed buprenorphine formulation, including its degradation products as peaks A and B.

FIG. 4A and FIG. 4B show MS spectra of Peak A.

FIG. 4C shows an MS spectrum of Peak A with the m/z 933 ion extracted at a lower intensity.

FIG. 5 shows an MS spectrum of Peak B.

FIG. 6 shows change in total degradation over 24 months at 25° C. and 30° C. for formulation K.

FIG. 7 shows additive long-term stability for formulation K at 25° C. and 30° C.

FIG. 8 shows change in weight loss adjusted potency over 24 months for formulation K at 25° C. and 30° C.

FIG. 9 shows predicted change in potency over 36 months at 25° C. versus coded levels for amounts of BHA and BHT used in formulations A-P.

FIG. 10 shows predicted change in potency versus total target amount of BHA and BHT (wt/wt %) in formulations A-P.

FIG. 11 shows predicted change in potency versus total target amount of BHA and BHT (wt/wt %) in formulations A-P for each tube type (PF113 or PF413).

FIG. 12 shows predicted change in potency versus nitrogen (Yes (Y) or No (N)) and overlaid with tube type (PF113 or PF413) for formulations A, B, N and O.

### DETAILED DESCRIPTION

The present disclosure relates to formulations, methods, uses and devices containing buprenorphine or salt thereof. This disclosure provides methods of providing pain relief to animals, for example cats. The present disclosure further relates to stable formulations of buprenorphine. This disclosure further provides methods of storing and shipping stable formulations of buprenorphine and related methods of use for treating or controlling pain such as postoperative pain associated with surgical procedures in cats.

Various methods of use relate to treating, controlling, and/or preventing pain in animals (particularly cats) in need thereof. These methods can comprise administering a pharmaceutical composition comprising a therapeutically effective amount of buprenorphine or salt thereof to the animal. The pharmaceutical composition can be formulated as a liquid formulation (and, for example, is preferably not in the form of patch). Administration can be transdermal as described herein (e.g., is applied to the dorsal cervical region of the animal). Further, the pain treated, controlled, and/or prevented can be postoperative pain associated with surgical procedures (e.g., in cats).

Applicant has unexpectedly and surprisingly discovered that formulating buprenorphine with certain additives (e.g., butylated hydroxyanisole (BHA), butylated hydroxytoluene (BHT) or a combination thereof) in low concentrations advantageously reduces or eliminates degradation of the active ingredient providing for long-term formulation stability of at least 6 months, at least 9 months, at least 12 months, at least 18 months, or at least 24 months. It has been further unexpectedly discovered that these combinations of additives provide for long-term formulation stability even when not refrigerated and stored at room temperature (approximately 25° C.) conditions. Accordingly, the present invention provides for improved formulations of buprenorphine that have greatly enhanced shelf life and that do not require cold chain storage and shipping. The formulations of the present disclosure comprising buprenorphine are shown to be stable at ambient and higher temperatures for extended periods.

The formulations may comprise any suitable amount of buprenorphine or salt thereof. For example, the concentration of buprenorphine or salt thereof may be about 5 mg/mL or more, about 10 mg/mL or more, about 15 mg/mL or more, about 20 mg/mL or more, about 25 mg/mL or more, about 30 mg/mL or more, about 35 mg/mL or more, or about 40 mg/mL or more. In some embodiments, the concentration of buprenorphine or salt thereof is from about 5 mg/mL to about 40 mg/mL, from about 10 mg/mL to about 35 mg/mL, or from about 10 mg/mL to about 30 mg/mL. In further embodiments, the concentration of buprenorphine or salt thereof is about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL. In some embodiments, the buprenorphine is present as a salt, for example, buprenorphine hydrochloride.

The buprenorphine formulations may optionally comprise a penetration enhancer, particularly in those formulations intended for transdermal administration. Penetration enhancers may exert their effect by disrupting the packing of skin lipids and thus altering the barrier function of the stratum corneum, changing the partitioning of the drug at the stratum corneum-epidermis interface, and/or altering the thermodynamic properties of the drug.

The penetration enhancer may be present in any suitable amount. For example, the concentration of the penetration enhancer may be about 20 mg/mL or more, about 30 mg/mL or more, about 40 mg/mL or more, about 50 mg/mL or more, or about 60 mg/mL or more. In some embodiments, the concentration of the penetration enhancer is from about 20 mg/mL to about 70 mg/mL, from about 30 mg/mL to about 60 mg/mL, or from about 40 mg/mL to about 60 mg/mL. In other embodiments, the concentration of the penetration enhancer is about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL. An example of a suitable penetration enhancer can comprise padimate O.

In some embodiments, the buprenorphine formulations are liquid at standard room temperature and pressure. This aids in transdermal administration of the formulation onto the skin of the subject as well as in other routes of administration.

The liquid formulations typically include a solvent. For example, a volatile solvent may be employed to aid in the application and absorption of the formulation during transdermal administration. Suitable volatile solvents include alcohols, such as isopropanol, ethanol, and combinations thereof. In some embodiments, the solvent is ethanol (dehydrated alcohol).

In accordance with the present invention, the buprenorphine formulations comprise at least one of certain additives selected from the group consisting of butylated hydroxyanisole (BHA), butylated hydroxytoluene (BHT) and combinations thereof. Addition of one or more of these additives have been found to enhance long term storage stability and/or eliminate the need for refrigerated storage to maintain stability. In some embodiments, the formulation contains a combination of BHA and BHT. In some embodiments, the antioxidants in the formulation consist or consist essentially (i.e., constituting >90 wt. % or even 95 wt. % of the total amount of antioxidant) of BHA and BHT. This combination of additives provides for excellent formulation stability over a wide range of temperatures and storage conditions.

The concentration of the at least one additive (BHA and/or BHT) may be about 0.1 mg/mL or more, about 0.2 mg/mL or more, about 0.3 mg/mL or more, about 0.4 mg/mL or more, about 0.5 mg/mL or more, about 0.6 mg/mL or more, about 0.8 mg/mL or more, about 0.9 mg/mL or more, about 1.0 mg/mL or more, about 1.1 mg/mL or more, about 1.2 mg/mL or more, about 1.3 mg/mL or more, about 1.4 mg/mL or more, or about 1.5 mg/mL or more. In some embodiments, the concentration of the at least one additive is from about 0.1 mg/mL to about 1.5 mg/mL, from about 0.1 mg/mL to about 1.0 mg/mL, from about 0.2 mg/mL to about 1.0 mg/mL, from about 0.2 mg/mL to about 0.9 mg/mL, from about 0.3 mg/mL to about 0.9 mg/mL, from about 0.4 mg/mL to about 0.9 mg/mL, from about 0.5 mg/mL to about 0.9 mg/mL, or from about 0.6 mg/mL to about 0.8 mg/mL.

The concentration of BHA is typically about 0.04 mg/mL or more, about 0.1 mg/mL or more, about 0.2 mg/mL or more, about 0.3 mg/mL or more, about 0.4 mg/mL or more, about 0.5 mg/mL or more, about 0.7 mg/mL or more, or about 1.0 mg/mL or more. For example, in some embodiments, the concentration of BHA is from about 0.04 mg/mL to about 1.0 mg/mL, from about 0.1 mg/mL to about 1.0 mg/mL, from about 0.2 mg/mL to about 1.0 mg/mL, from about 0.2 mg/mL to about 0.7 mg/mL, from about 0.3 mg/mL to about 0.7 mg/mL, from about 0.3 mg/mL to about 0.6 mg/mL, from about 0.3 mg/mL to about 0.5 mg/mL, or from about 0.3 mg/mL to about 0.4 mg/mL of BHA.

The concentration of BHT is typically about 0.1 mg/mL or more, about 0.2 mg/mL or more, about 0.3 mg/mL or more, about 0.4 mg/mL or more, about 0.5 mg/mL or more, about 0.7 mg/mL or more, or about 1.0 mg/mL or more. For example, in some embodiments, the concentration of BHT is from about 0.1 mg/mL to about 1.0 mg/mL, from about 0.2 mg/mL to about 1.0 mg/mL, from about 0.2 mg/mL to about 0.7 mg/mL, from about 0.3 mg/mL to about 0.7 mg/mL, from about 0.3 mg/mL to about 0.6 mg/mL, from about 0.3 mg/mL to about 0.5 mg/mL, or from about 0.3 mg/mL to about 0.4 mg/mL.

In formulations containing both BHA and BHT additives, the weight ratio of BHT to BHA may be about 1.1:1 or greater, about 2:1 or greater, about 5:1 or greater, or about 10:1 or greater. As discussed in detail in Example 1, the presence of both BHA and BHT, particularly when BHT is present in an amount at least equivalent to that of BHA, slows the rate of decrease of both of these compounds and provides for exceptional formulation stability. In some embodiments, the weight ratio of BHT to BHA is about 1:1.

In some embodiments, the formulation comprises buprenorphine hydrochloride, padimate O, ethanol, and a combination of BHA and BHT additives. In such embodiments, the concentration buprenorphine hydrochloride is from about 10 mg/mL to about 30 mg/mL; the concentration of padimate O is from about 40 mg/mL to about 60 mg/mL; and the concentration of the combination of BHA and BHT is from about 0.5 mg/mL to about 0.9 mg/mL, wherein the weight ratio of BHT to BHA is about 1:1 or greater; and a solvent comprising ethanol.

Stability of the buprenorphine in the present formulations can be evaluated by determining relative decreases in the concentrations of buprenorphine degradation products. These buprenorphine degradation products are identified and further described in Example 1. In particular, buprenorphine degradation products are pseudo buprenorphine (a dimer of buprenorphine) and/or a positional isomer thereof.

In some embodiments, the formulation is stable such that the concentration of buprenorphine degradation products in the formulation is less than that of an otherwise identical formulation not containing BHA and/or BHT after storage for up to 3 months, 6 months, 9 months, 12 months or 24 months at 25° C. and higher temperatures.

In various embodiments such as these, the concentration of buprenorphine degradation products in the formulation after 3 months of storage at 25° C. can be about 0.05 mg/mL or less, about 0.04 mg/mL or less, about 0.03 mg/mL or less, about 0.02 mg/mL or less, about 0.01 mg/mL or less, about 0.009 mg/mL or less, about 0.008 mg/mL or less, about 0.007 mg/mL or less, about 0.006 mg/mL or less, about 0.005 mg/mL or less, about 0.004 mg/mL or less, about 0.003 mg/mL or less, about 0.002 mg/mL or less, or about 0.001 mg/mL or less. In this and other embodiments, the concentration of buprenorphine degradation products in the formulation after 3 months of storage at 30° C. can be about 0.08 mg/mL or less, about 0.07 mg/mL or less, about 0.06 mg/mL or less, about 0.05 mg/mL or less, about 0.04 mg/mL or less, about 0.03 mg/mL or less, about 0.02 mg/mL or less, about 0.01 mg/mL or less, about 0.009 mg/mL or less, about 0.008 mg/mL or less, about 0.007 mg/mL or less, about 0.006 mg/mL or less, about 0.005 mg/mL or less, about 0.004 mg/mL or less, about 0.003 mg/mL or less, about 0.002 mg/mL or less, or about 0.001 mg/mL or less. In further embodiments, the concentration of buprenorphine degradation products in the formulation after 3 months of storage at 40° C. can be about 3 mg/mL or less, about 2.5 mg/mL or less, about 2 mg/mL or less, about 1.5 mg/mL or less, about 1 mg/mL or less, about 0.90 mg/mL or less, about 0.80 mg/mL or less, about 0.70 mg/mL or less, about 0.60 mg/mL or less, about 0.50 mg/mL or less, about 0.40 mg/mL or less, about 0.30 mg/mL or less, about 0.20 mg/mL or less, about 0.10 mg/mL or less, about 0.09 mg/mL or less, about 0.08 mg/mL or less, about 0.07 mg/mL or less, about 0.06 mg/mL or less, about 0.05 mg/mL or less, about 0.04 mg/mL or less, about 0.03 mg/mL or less, about 0.02 mg/mL or less, or about 0.01 mg/mL or less. In still further embodiments, the concentration of buprenorphine degradation products in the formulation after 12 months of storage at 25° C. can be about 0.20 mg/mL or less, about 0.15 mg/mL or less, about 0.10 mg/mL or less, about 0.09 mg/mL or less, about 0.08 mg/mL or less, about 0.07 mg/mL or less, about 0.06 mg/mL or less, about 0.05 mg/mL or less, about 0.04 mg/mL or less, about 0.03 mg/mL or less, about 0.02 mg/mL or less, about 0.01 m or less g/mL, about 0.009 mg/mL or less, about 0.008 mg/mL or less, about 0.007 mg/mL or less, about 0.006 mg/mL or less, about 0.005 mg/mL or less, about 0.004 mg/mL or less, about 0.003 mg/mL or less, about 0.002 mg/mL or less, or about 0.001 mg/mL or less.

The present formulations can be packaged as unit doses. The unit doses can be any volume. For example, the unit dose may be from about 0.5 mL to about 1.0 mL. In some embodiments, the unit dose is about 0.7 mL. In other embodiments, the unit dose is about 1.0 mL.

The unit doses may be contained in any appropriate vessel. In some embodiments, the vessel is a tube. In some embodiments, the tube is an aluminum and polymer laminate tube, for example, a PF113 tube from Neopac US, Inc. (Wilson, North Carolina).

The dosage of buprenorphine may be in the range of from about 1 mg/kg to about 10 mg/kg, from about 2 mg/kg to about 8 mg/kg, or from about 2.5 mg/kg to about 7 mg/kg.

The disclosure is also directed to a method for storing the formulations described herein comprising storing the formulation at a temperature greater than 5° C. The formulation may advantageously be stored at a temperature from about 5° C. to about 25° C. The formulation may be stored for up to 6 months, 9 months, 12 months, 24 months, 36 months or longer.

Another aspect of the disclosure is a method for shipping the formulation described herein comprising shipping the formulation at a temperature greater than 5° C. The formulation may be shipped at a temperature from about 5° C. to about 25° C. By providing a formulation that maintains stability and efficacy during prolonged storage and shipping operations, even in the absence of refrigeration, handling and administration of the formulation is simplified.

The disclosure is further directed to a method for reducing pain in a mammal subject in need thereof comprising administering to the subject the formulations described herein. The pain may be postoperative pain associated with a surgical procedure. The mammal subject may be any mammal. In some embodiments, the mammal subject is a companion animal, such as a feline or canine.

The formulation is typically administered at a temperature similar to or approximately the same temperature at which it is stored, for example, from about 5° C. to about 25° C., from about 10° C. to about 25° C., or from about 15° C. to about 25° C. For example, the formulation may be administered at a temperature within 5° C. of the temperature at which the formulation is stored prior to administration. Because the formulations described herein are stable over a wide range of temperatures and do not require refrigeration, the formulation does not need to be heated prior to administration. The present invention provides for improved formulations of buprenorphine that have a longer shelf-life of 9 months, 12 months, 24 months, 36 months or longer and do not require cold chain storage and shipping (e.g., can be stored at about 25° C.).

The formulation may be administered transdermally, intravenously, intramuscularly, subcutaneously, epidurally, intrathecally, or sublingually. In some embodiments, the formulation is administered transdermally.

The present formulations may be packaged in applicators comprising a reservoir for containing the formulation and an applicator tip for applying the formulation to the subject. The applicator tip may be designed to make it convenient for the product to reach the skin through the hair without having to shave the application spot. The present formulations may be packaged in a unit dose pack. It is preferred that the package comprise a single dose of the formulation. The packaging may be of any suitable material, such as an aluminum polymer laminated tube, that prevents the volatilization of the solvent or ingress of oxygen. The present disclosure provides a device for use in a method of treating pain in a companion animal. Said device comprises a reservoir for storing a TBS formulation as described herein at room temperature and an applicator for administering the formulation to the skin of said animal. The applicator is preferably adapted to apply the formulation through the coat of a companion animal.

Treatments may be administered topically to the dorsal cervical region (base of the skull). For example, the applicator tube tip may be placed directly onto the skin at the application site by parting the hair, if necessary, and the entire dose volume administered at a single location. Alternatively, the dose volume may be distributed over two or more sites. The formulation may be applied by gently holding the subject (e.g. cat) to prevent shaking or rubbing during application. Contacting the applicator with the skin and dispensing the contents. Remove the applicator from the application site while attempting to avoid contact with the hair. Contact with the site of application should be avoided while the formulation dries (approx. 30 minutes after administration).

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “about” when used in connection with a measurable numerical variable, refers to the indicated value of the variable and to all values of the variable that are within the experimental error of the indicated value or within ±10 percent of the indicated value, whichever is greater.

The term “effective amount” refers to an amount which gives the desired benefit to the subject and includes administration for both treatment and control. The amount may vary from one individual subject to another and will depend upon a number of factors, including the overall physical condition of the subject and the severity of the underlying cause of the condition to be treated, concomitant treatments, and the amount of compound of the disclosure used to maintain desired response at a beneficial level.

An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, infection, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances. An effective amount of the present disclosure, the active ingredient treatment dosage, may range from, for example, 0.5 mg to 100 mg. Specific amounts can be determined by the skilled person. Although these dosages are based on a subject having a mass of about 1 kg to about 20 kg, the diagnostician will be able to determine the appropriate dose for a subject whose mass falls outside of this weight range. An effective amount of the present disclosure, the active ingredient treatment dosage, may range from, for example, 0.1 mg to 10 mg/kg of the subject. The dosing regimen is expected to be daily, weekly, or monthly administration.

The terms “subject” and “patient” refers includes non-human mammalian animals, such as dogs, cats, mice, rats, guinea pigs, rabbits, ferrets, cows, horses, sheep, goats, and pigs. More particular subjects are mammalian pets or companion animals, such as dogs and cats and also mice, rats, guinea pigs, ferrets, and rabbits.

The terms “treating,” “to treat,” “treated,” or “treatment,” include without limitation restraining, slowing, stopping, reducing, ameliorating, reversing the progression or severity of an existing symptom, or preventing a disorder, condition, or disease. A treatment may be applied or administered therapeutically.

The skilled artisan will appreciate that certain of the compounds of the present disclosure exist as isomers. All stereoisomers of the compounds of the disclosure, including geometric isomers, enantiomers, and diastereomers, in any ratio, are contemplated to be within the scope of the present disclosure. The skilled artisan will also appreciate that certain of the compounds of the present disclosure exist as tautomers. All tautomeric forms the compounds of the disclosure are contemplated to be within the scope of the present disclosure.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In an embodiment, a formulation can comprise buprenorphine or salt thereof, and at least one additive selected from the group comprising butylated hydroxyanisole (BHA), butylated hydroxytoluene (BHT) and combinations thereof.

The concentration of buprenorphine or salt thereof can be about 5 mg/mL or more, about 10 mg/mL or more, about 15 mg/mL or more, about 20 mg/mL or more, about 25 mg/mL or more, about 30 mg/mL or more, about 35 mg/mL or more, or about 40 mg/mL or more. The concentration of buprenorphine or salt thereof can be from about 5 mg/mL to about 40 mg/mL, from about 10 mg/mL to about 35 mg/mL, or from about 10 mg/mL to about 30 mg/mL. The concentration of buprenorphine or salt thereof can be about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL.

The buprenorphine or salt thereof can comprise buprenorphine hydrochloride.

The formulation can further comprise a penetration enhancer. The concentration of the penetration enhancer can be about 20 mg/mL or more, about 30 mg/mL or more, about 40 mg/mL or more, about 50 mg/mL or more, or about 60 mg/mL or more. The concentration of the penetration enhancer can be from about 20 mg/mL to about 70 mg/mL, from about 30 mg/mL to about 60 mg/mL, or from about 40 mg/mL to about 60 mg/mL. The concentration of the penetration enhancer can be about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, or about 60 mg/mL. The penetration enhancer can comprise padimate O.

The formulation can further comprise a solvent. The solvent can be a volatile solvent. The solvent can be selected from the group consisting of isopropanol, ethanol, and combinations thereof. The solvent can comprise ethanol.

The formulation can be stable such that the concentration of buprenorphine degradation products in the formulation can be less than that of an otherwise identical formulation not containing BHA and/or BHT after storage for up to 3 months, 6 months, 9 months, 12 months or 24 months at 25° C.

The concentration of buprenorphine degradation products in the formulation after 3months of storage at 25° C. can be about 0.05 mg/mL or less, about 0.04 mg/mL or less, about 0.03 mg/mL or less, about 0.02 mg/mL or less, about 0.01 mg/mL or less, about 0.009 mg/mL or less, about 0.008 mg/mL or less, about 0.007 mg/mL or less, about 0.006 mg/mL or less, about 0.005 mg/mL or less, about 0.004 mg/mL or less, about 0.003 mg/mL or less, about 0.002 mg/mL or less, or about 0.001 mg/mL or less.

The concentration of buprenorphine degradation products in the formulation after 3months of storage at 30° C. can be about 0.08 mg/mL or less, about 0.07 mg/mL or less, about 0.06 mg/mL or less, about 0.05 mg/mL or less, about 0.04 mg/mL or less, about 0.03 mg/mL or less, about 0.02 mg/mL or less, about 0.01 mg/mL or less, about 0.009 mg/mL or less, about 0.008 mg/mL or less, about 0.007 mg/mL or less, about 0.006 mg/mL or less, about 0.005 mg/mL or less, about 0.004 mg/mL or less, about 0.003 mg/mL or less, about 0.002 mg/mL or less, or about 0.001 mg/mL or less.

The concentration of buprenorphine degradation products in the formulation after 3 months of storage at 40° C. can be about 3 mg/mL or less, about 2.5 mg/mL or less, about 2 mg/mL or less, about 1.5 mg/mL or less, about 1 mg/mL or less, about 0.90 mg/mL or less, about 0.80 mg/mL or less, about 0.70 mg/mL or less, about 0.60 mg/mL or less, about 0.50 mg/mL or less, about 0.40 mg/mL or less, about 0.30 mg/mL or less, about 0.20 mg/mL or less, about 0.10 mg/mL or less, about 0.09 mg/mL or less, about 0.08 mg/mL or less, about 0.07 mg/mL or less, about 0.06 mg/mL or less, about 0.05 mg/mL or less, about 0.04 mg/mL or less, about 0.03 mg/mL or less, about 0.02 mg/mL or less, or about 0.01 mg/mL or less.

The concentration of buprenorphine degradation products in the formulation after 12 months of storage at 25° C. can be about 0.20 mg/mL or less, about 0.15 mg/mL or less, about 0.10 mg/mL or less, about 0.09 mg/mL or less, about 0.08 mg/mL or less, about 0.07 mg/mL or less, about 0.06 mg/mL or less, about 0.05 mg/mL or less, about 0.04 mg/mL or less, about 0.03 mg/mL or less, about 0.02 mg/mL or less, about 0.01 m or less g/mL, about 0.009 mg/mL or less, about 0.008 mg/mL or less, about 0.007 mg/mL or less, about 0.006 mg/mL or less, about 0.005 mg/mL or less, about 0.004 mg/mL or less, about 0.003 mg/mL or less, about 0.002 mg/mL or less, or about 0.001 mg/mL or less.

The buprenorphine degradation products can comprise a polymer of buprenorphine.

The buprenorphine degradation products can comprise a dimer of buprenorphine and/or a positional isomer thereof.

The concentration of the at least one additive can be about 0.1 mg/mL or more, about 0.2 mg/mL or more, about 0.3 mg/mL or more, about 0.4 mg/mL or more, about 0.5 mg/mL or more, about 0.6 mg/mL or more, about 0.8 mg/mL or more, about 0.9 mg/mL or more, about 1.0 mg/mL or more, about 1.1 mg/mL or more, about 1.2 mg/mL or more, about 1.3 mg/mL or more, about 1.4 mg/mL or more, or about 1.5 mg/mL or more.

The concentration of the at least one additive can be from about 0.1 mg/mL to about 1.5 mg/mL, from about 0.1 mg/mL to about 1.0 mg/mL, from about 0.2 mg/mL to about 1.0 mg/mL, from about 0.2 mg/mL to about 0.9 mg/mL, from about 0.3 mg/mL to about 0.9 mg/mL, from about 0.4 mg/mL to about 0.9 mg/mL, from about 0.5 mg/mL to about 0.9 mg/mL, or from about 0.6 mg/mL to about 0.8 mg/mL.

The formulation can comprise BHA. The formulation can comprise BHT. The formulation can comprise BHA and BHT or the antioxidants in the formulation consist or consist essentially (i.e., constituting >90 wt. % or even 95 wt. % of the total amount of antioxidant) of BHA and BHT. The concentration of BHA can be 0.04 mg/mL or more, about 0.1 mg/mL or more, about 0.2 mg/mL or more, about 0.3 mg/mL or more, about 0.4 mg/mL or more, about 0.5 mg/mL or more, about 0.7 mg/mL or more, or about 1.0 mg/mL or more. The concentration of BHA can be from about 0.04 mg/mL to about 1.0 mg/mL, from about 0.1 mg/mL to about 1.0 mg/mL, from about 0.2 mg/mL to about 1.0 mg/mL, from about 0.2 mg/mL to about 0.7 mg/mL, from about 0.3 mg/mL to about 0.7 mg/mL, from about 0.3 mg/mL to about 0.6 mg/mL, from about 0.3 mg/mL to about 0.5 mg/mL, or from about 0.3 mg/mL to about 0.4 mg/mL of BHA. The concentration of BHT can be about 0.1 mg/mL or more, about 0.2 mg/mL or more, about 0.3 mg/mL or more, about 0.4 mg/mL or more, about 0.5 mg/mL or more, about 0.7 mg/mL or more, or about 1.0 mg/mL or more. The concentration of BHT can be from about 0.1 mg/mL to about 1.0 mg/mL, from about 0.2 mg/mL to about 1.0 mg/mL, from about 0.2 mg/mL to about 0.7 mg/mL, from about 0.3 mg/mL to about 0.7 mg/mL, from about 0.3 mg/mL to about 0.6 mg/mL, from about 0.3 mg/mL to about 0.5 mg/mL, or from about 0.3 mg/mL to about 0.4 mg/mL.

The weight ratio of BHT to BHA can be about 1.1:1 or greater, about 2:1 or greater, about 5:1 or greater, or about 10:1 or greater.

The formulation can comprise:
from about 10 mg/mL to about 30 mg/mL buprenorphine hydrochloride;
from about 40 mg/mL to about 60 mg/mL padimate O;
from about 0.5 mg/mL to about 0.9 mg/mL of a combination of BHA and BHT, wherein the weight ratio of BHT to BHA is about 1:1 or greater; and
a solvent comprising ethanol.

The formulation can be packaged as a unit dose. The unit dose can be contained in an aluminum and polymer laminate tube. The unit dose can be from about 0.5 ml to about 1.0 ml. The unit dose can be about 1.0 ml.

In an embodiment, a method for storing a formulation described herein can comprise storing the formulation at a temperature greater than 5° C. The formulation can be stored at a temperature from about 5° C. to about 25° C. The formulation can be stored for up to 12 months, 24 months or 36 months. The formulation can be stored at about 25° C.

In an embodiment, a method for shipping the formulation described herein can comprise shipping the formulation at a temperature greater than 5° C. The formulation can be shipped at a temperature from about 5° C. to about 25° C. The formulation can be shipped at about 25° C.

In an embodiment, a method for reducing pain in a mammal subject in need thereof can comprise administering to the subject a formulation described herein. The formulation can be administered transdermally, intravenously, intramuscularly, subcutaneously, epidurally, intrathecally, or sublingually. The formulation can be administered transdermally.

The pain can be postoperative pain associated with a surgical procedure.

The subject can be a feline.

The formulation can be administered at a temperature from about 5° C. to about 25° C., from about 10° C. to about 25° C., or from about 15° C. to about 25° C.

The formulation can be administered at a temperature within 5° C. of the temperature at which the formulation can be stored prior to administration.

The formulation can be administered at approximately the same temperature at which the formulation can be stored prior to administration.

The formulation can be not heated prior to administration.

### EXAMPLES

The present invention has multiple aspects, illustrated by the following non-limiting examples.

### Example 1

Sixteen formulation batches of buprenorphine HCl (20 mg/mL buprenorphine, 8 mg/0.4 mL per tube) containing varying amounts of additives BHA and BHT were prepared according to the Design of Experiments (DOE) described in Table 1 and Table 2 below. Each formulation also contained 50 mg/ml of Padimate-O (penetration enhancer) and dehydrated ethanol solvent (Q.S.).

TABLE 1
Experimental Design Factors

| | | Levels | | | |
|---|---|---|---|---|---|
| Factor | Description | 0 | L | M | H |
| BHA | Concentration | 0% (wt/wt) | 0.005% (wt/wt) | 0.02% (wt/wt) | 0.05% (wt/wt) |
| | (% w/w) of BHA in | 0 (mg/mL) | 0.0404(mg/mL) | 0.162(mg/mL) | 0.404 (mg/mL) |
| | Formulation | | | | |
| BHT | Concentration | 0 | 0.02% (wt/wt) | 0.05% (wt/wt) | 0.09% (wt/wt) |
| | (% w/w) of BHT in | | 0.162 (mg/mL) | 0.404 (mg/mL) | 0.727 (mg/mL) |
| | Formulation | | | | |
| Nitrogen | Use of Nitrogen | Y = Yes | N = No | | |
| | Blanket during | | | | |
| | filling | | | | |
| Tube Type | Type of tube used | Tube = PF113 | Tube = PF413 | | |

TABLE 2
Prepared Formulations DOE Factor Combinations

| | | | | | | BHA | BHT |
|---|---|---|---|---|---|---|---|
| | | BHA | | | | Formulation | Formulation |
| Batch | Formula | Level | BHT Level | Nitrogen | Tube Type | Target (%) | Target (%) |
| A | 00Y113 | 0 | 0 | Y | PF113 | 0 | 0 |
| B | 00Y413 | 0 | 0 | Y | PF413 | 0 | 0 |
| C | 0LN113 | 0 | L | N | PF113 | 0 | 0.02 |
| D | 0HN113 | 0 | H | N | PF113 | 0 | 0.09 |
| E | 0HN413 | 0 | H | N | PF413 | 0 | 0.09 |
| F | L0N113 | L | 0 | N | PF113 | 0.005 | 0 |
| G | LMN113 | L | M | N | PF113 | 0.005 | 0.05 |
| H | MHN113 | M | H | N | PF113 | 0.02 | 0.09 |
| I | H0N113 | H | 0 | N | PF113 | 0.05 | 0 |
| J | H0N413 | H | 0 | N | PF413 | 0.05 | 0 |
| K | HMN113 | H | M | N | PF113 | 0.05 | 0.05 |
| L | HHN113 | H | H | N | PF113 | 0.05 | 0.09 |
| M | LMN113 | L | M | N | PF113 | 0.005 | 0.05 |
| N | 00N113 | 0 | 0 | N | PF113 | 0 | 0 |
| O | 00N413 | 0 | 0 | N | PF413 | 0 | 0 |
| P | HHN413 | H | H | N | PF413 | 0.05 | 0.09 |

Key for Formula Label
First Digit BHA - High (H), Mid (M), None (0)
2nd Digit BHA - High (H), Mid (M), None (0)
3rd Digit Nitrogen - Yes (Y) or No (N)
Last 3 Digits Tube Type - PF113 or PF413

The prepared formulation batches were evaluated for stability according to the testing plan shown in Table 3 below.

TABLE 3
Stability Testing Plan

| | Time Points (Months) | | | | | | | | |
|---|---|---|---|---|---|---|---|---|---|
| Storage Conditions | Initial (0) | 1 | 2 | 3 | 6 | 9 | 12 | 18 | 24 |
| 5 C. | x | | | x | x | | x | | |
| 25 C./60% RH | | | | x | x | x | x | x | x |
| 30 C./65% RH | | x | x | x | x | x | * | * | |
| 40 C./5% RH | | x | x | x | x | | | | |

*optional testing, if determined to be necessary

At each time point, the properties shown in Table 4 were collected and used in the evaluation. The 0-month data was collected initially after packaging and those results are used as the common initial time results for all temperature/relative humidity storage conditions.

TABLE 4
Properties Measured
Test Parameter

| | |
|---|---|
| Appearance | Clear and colorless to yellow |
| | solution free of visible matter |
| Assay | Actual content (mg/mL) and |
| Buprenorphine | percent of target = (actual content)/(target content) |
| | target content = 20 mg/mL |
| BHA | Concentration (mg/mL) and percent |
| | of starting target amount |
| BHT | Concentration (mg/mL) and percent |
| | of starting target amount |
| Related | Individual Related substances recorded (mg/mL) |
| Substances | Total Related substances recorded (mg/mL) |
| (Degradation | |
| Products) | |

The related substances measured (as noted in Table 4) are the two most prominent peaks (“Degradation Product 1” and “Degradation Product 2”) that have been shown to increase over time when the product is exposed to oxidative conditions. As discussed further below, these peaks correspond to two degradation products which are consistent with pseudo buprenorphine and a position isomer of pseudo buprenorphine. The Total Related Substances used for analysis in this example is the sum of those two individual related substances.

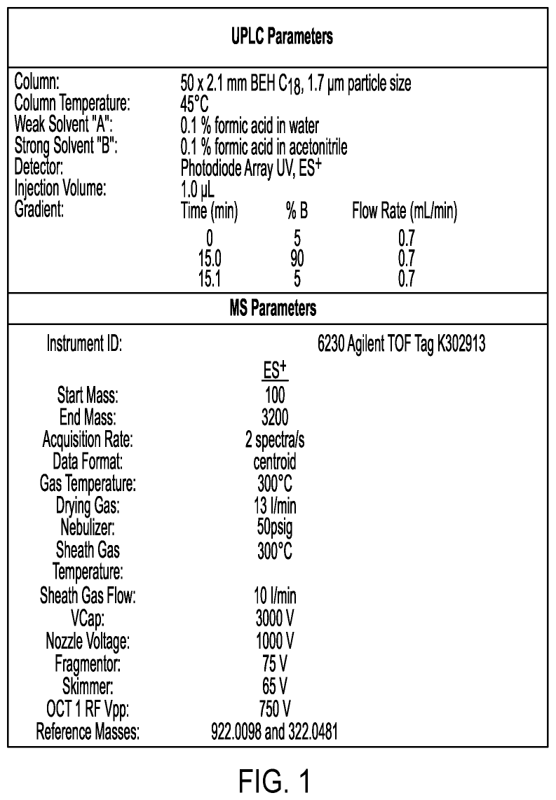

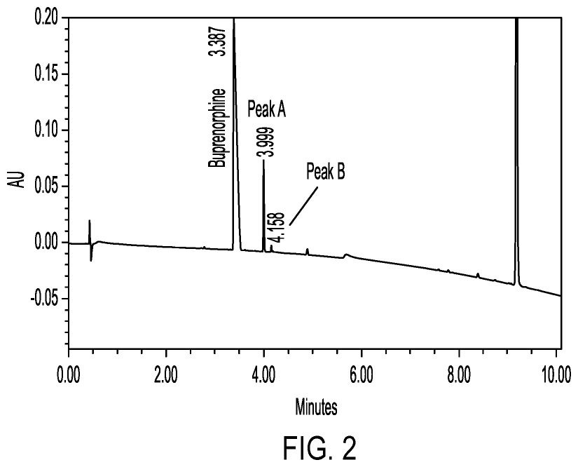

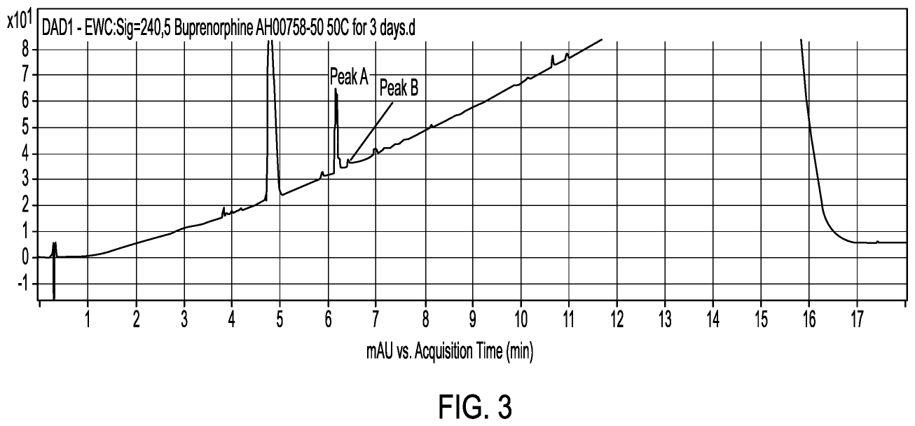

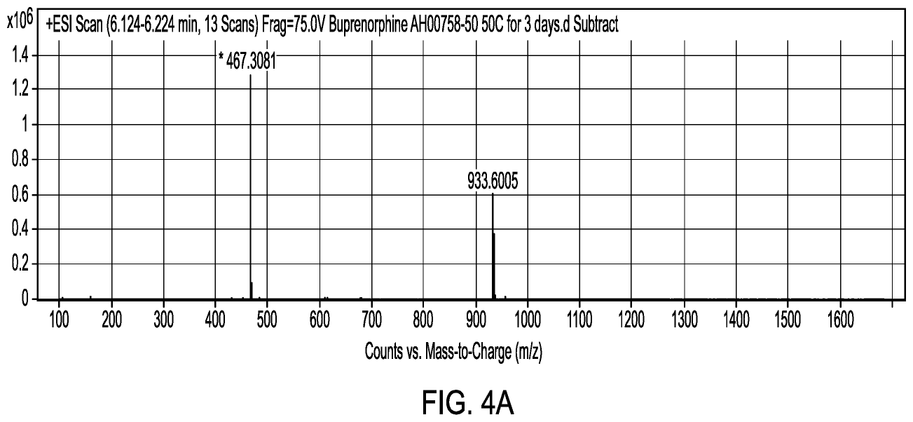

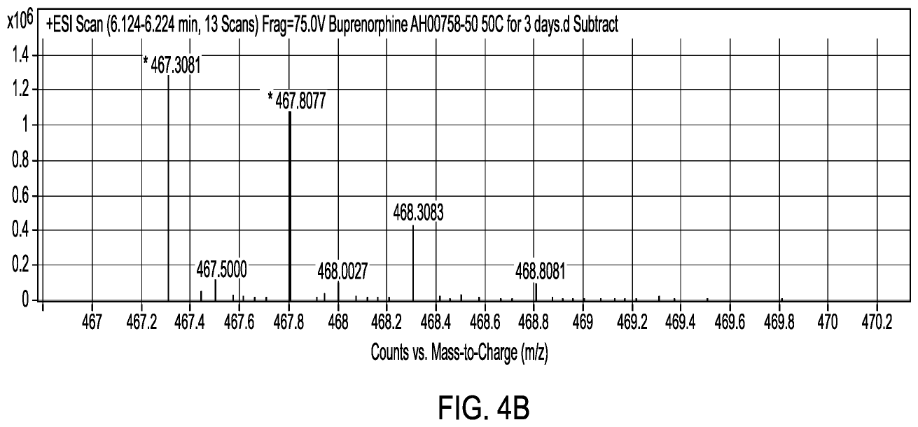

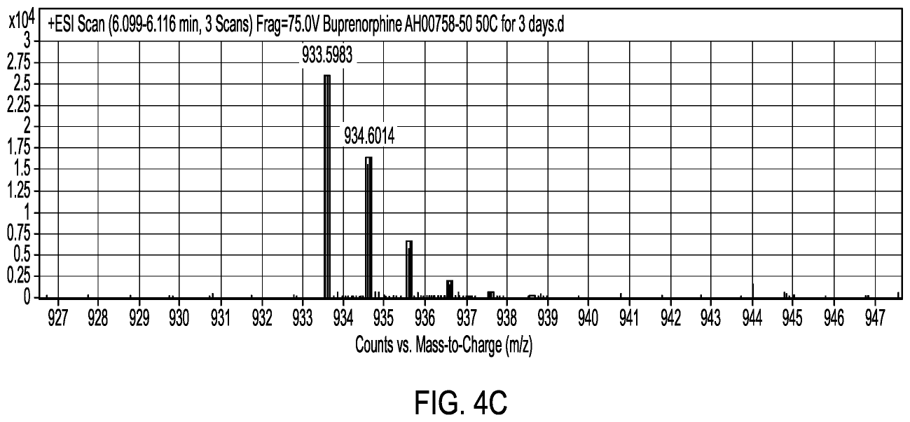

“Degradation Product 1” and “Degradation Product 2” were identified by UPLC-MS method (FIG. 1). A control formulation containing buprenorphine and no BHA or BHT was stressed at 50° C. for 3 days. The sample was prepared for analysis by diluting 50 μL of the formulation with 950 μL of 50/50 water/acetonitrile for UPLC and Aligent TOF MS. Peaks A and B in the UPLC chromatogram correspond to “Degradation Product 1” and “Degradation Product 2,” respectively (FIG. 2-3). The MS spectra for peak A resulted in a m/z 467 doubly charged ion and a m/z 933 ion (FIG. 4A-4B). The m/z 933 ion was extracted at a lower intensity in order to obtain a more accurate mass value (FIG. 4C).

After analysis, the best empirical formula fit was C₅₈H₈₀N₂O₈. Peak A is consistent with pseudo buprenorphine, a dimer of buprenorphine with a chemical formula of C₅₈H₈₀N₂O₈ and an exact mass of 932.5915. The structure of pseudo buprenorphine is shown below:

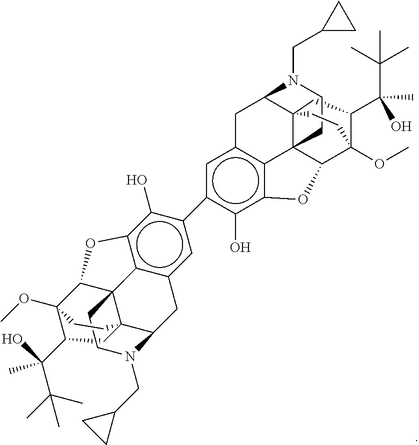

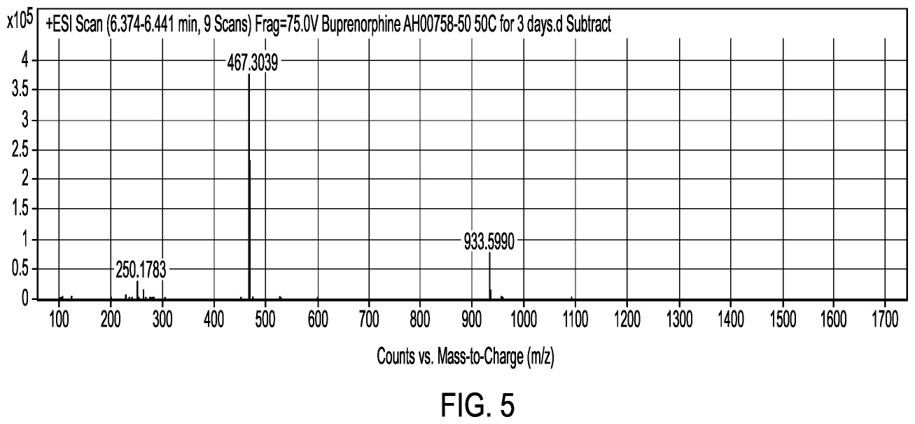

Peak B appears to be isomeric with Peak A based on its MS spectrum (FIG. 5). Peak B is consistent with a positional isomer of pseudo buprenorphine.

For comparison, buprenorphine has a chemical formula of C₂₉H₄₁NO₄ with an exact mass of 467.3036, with its structure shown below:

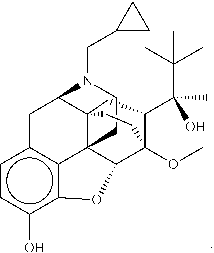

40 tubes per batch of buprenorphine formulation listed in Table 2 were placed in a chamber (10 tubes per storage condition) to monitor for stability over time and analyzed for potency (i.e., concentration of buprenorphine) and degradation products according to the analytical method and stability test plan (Table 3). The stability data is shown in Tables 5-8 below.

TABLE 5
Stability Data - Potency and Total Degradation Products. Shaded cells indicate that
data was not collected at that time point and condition for the analytical property.

| | | Potency | | | | Total Degradation | | | |
|---|---|---|---|---|---|---|---|---|---|
| | | (mg/mL) | | | | Products (mg/mL) | | | |
| | | Storage Condition | | | | Storage Condition | | | |
| | Time | | 25° C./ | 30° C./ | 40° C./ | | 25° C./ | 30° C./ | 40° C./ |
| Batch | (months) | 5° C. | 60% RH | 65% RH | 75% RH | 5° C. | 60% RH | 65% RH | 75% RH |
| A | 0 | 20.37 | 20.37 | 20.37 | 20.37 | 0.0705 | 0.0705 | 0.0705 | 0.0705 |
| | 1 | | | 19.59 | 18.59 | | | 0.6763 | 2.8393 |
| | 2 | | | 18.84 | 15.13 | | | 2.2979 | 7.6011 |
| | 3 | 19.92 | 19.06 | 18.17 | 12.77 | 0.1103 | 1.8034 | 4.0056 | 9.1089 |
| B | 0 | 20.46 | 20.46 | 20.46 | 20.46 | 0.0671 | 0.0671 | 0.0671 | 0.0671 |
| | 1 | | | 19.84 | 17.24 | | | 0.7761 | 4.5724 |
| | 2 | | | 18.70 | 13.61 | | | 2.8010 | 8.0193 |
| | 3 | 19.95 | 19.31 | 17.50 | 11.70 | 0.1200 | 1.7192 | 4.8256 | 9.3966 |
| C | 0 | 20.92 | 20.92 | 20.92 | 20.92 | 0.0420 | 0.0420 | 0.0420 | 0.0420 |
| | 1 | | | 20.62 | 19.64 | | | 0.1571 | 1.4773 |
| | 2 | | | 20.27 | 16.94 | | | 0.5052 | 6.0484 |
| | 3 | 20.73 | 20.60 | 19.89 | 14.30 | 0.0515 | 0.2851 | 1.2764 | 8.2282 |
| D | 0 | 21.00 | 21.00 | 21.00 | 21.00 | 0.0422 | 0.0422 | 0.0422 | 0.0422 |
| | 1 | | | 21.30 | 21.18 | | | 0.0515 | 0.1014 |
| | 2 | | | 21.06 | 20.67 | | | 0.0722 | 0.3278 |
| | 3 | 21.10 | 20.95 | 20.95 | 19.92 | 0.0441 | 0.0583 | 0.0773 | 0.9294 |
| | 6 | 20.99 | 20.93 | 20.63 | 16.52 | 0.0401 | 0.0730 | 0.2898 | 5.2245 |
| | 9 | | 20.96 | 20.72 | | | 0.0946 | 0.2701 | |
| | 12 | 21.14 | 20.93 | 20.50 | | 0.0402 | 0.1136 | 0.4898 | |
| E | 0 | 20.92 | 20.92 | 20.92 | 20.92 | 0.0413 | 0.0413 | 0.0413 | 0.0413 |
| | 1 | | | 2035 | 20.59 | | | 0.0956 | 0.1097 |
| | 2 | | | 20.75 | 20.00 | | | 0.0752 | 0.3853 |
| | 3 | 20.46 | 20.43 | 20.54 | 19.32 | 0.0416 | 0.1476 | 0.1013 | 1.0344 |
| F | 0 | 20.96 | 20.96 | 20.96 | 20.96 | 0.0507 | 0.0507 | 0.0507 | 0.0507 |
| | 1 | | | 20.42 | 19.64 | | | 0.2751 | 1.9989 |
| | 2 | | | 20.25 | 17.20 | | | 1.1322 | 5.6796 |
| | 3 | 20.60 | 20.32 | 19.38 | 14.42 | 0.0668 | 0.6192 | 2.3714 | 8.2299 |
| G | 0 | 20.93 | 20.93 | 20.93 | 20.93 | 0.0412 | 0.0412 | 0.0412 | 0.0412 |
| | 1 | | | 20.75 | 20.49 | | | 0.0563 | 0.1536 |
| | 2 | | | 20.67 | 20.16 | | | 0.0798 | 0.5820 |
| | 3 | 20.81 | 20.81 | 21.00 | 19.18 | 0.0433 | 0.0629 | 0.1012 | 2.0317 |
| | 6 | 20.68 | 20.93 | 20.58 | 15.46 | 0.0408 | 0.0825 | 0.1825 | 6.4161 |
| | 9 | | 21.11 | 20.52 | | | 0.1045 | 0.4184 | |
| | 12 | 21.21 | 20.84 | 20.25 | | 0.0398 | 0.1441 | 0.9713 | |
| H | 0 | 20.71 | 20.71 | 20.71 | 20.71 | 0.0411 | 0.0411 | 0.0411 | 0.0411 |
| | 1 | | | 20.61 | 20.76 | | | 0.0427 | 0.0595 |
| | 2 | | | 20.68 | 20.91 | | | 0.0477 | 0.0878 |
| | 3 | 20.84 | 20.96 | 21.03 | 20.76 | 0.0415 | 0.0421 | 0.0436 | 0.1044 |
| | 6 | 20.56 | 20.73 | 20.63 | 20.38 | 0.0404 | 0.0426 | 0.0534 | 0.2444 |
| | 9 | | 20.93 | 21.07 | | | 0.0420 | 0.0635 | |
| | 12 | 20.85 | 20.98 | 21.08 | | 0.0396 | 0.0516 | 0.0707 | |
| I | 0 | 20.76 | 20.76 | 20.76 | 20.76 | 0.0416 | 0.0416 | 0.0416 | 0.0416 |
| | 1 | | | 20.72 | 20.77 | | | 0.0588 | 0.1209 |
| | 2 | | | 20.53 | 20.23 | | | 0.0811 | 0.2163 |
| | 3 | 21.13 | 20.87 | 21.00 | 20.55 | 0.0419 | 0.0649 | 0.0921 | 0.2761 |
| | 6 | 20.46 | 20.48 | 20.47 | 19.77 | 0.0405 | 0.0787 | 0.1182 | 0.6099 |
| | 9 | | 20.78 | 20.70 | | | 0.0882 | 0.1584 | |
| | 12 | 20.90 | 20.63 | 20.55 | | 0.0397 | 0.1046 | 0.2089 | |
| J | 0 | 20.70 | 20.70 | 20.70 | 20.70 | 0.0422 | 0.0422 | 0.0422 | 0.0422 |
| | 1 | | | 20.66 | 20.56 | | | 0.0557 | 0.1034 |
| | 2 | | | 20.46 | 20.17 | | | 0.0757 | 0.1849 |
| | 3 | 21.09 | 21.05 | 20.81 | 20.29 | 0.0431 | 0.0610 | 0.0825 | 0.2713 |
| | 6 | 20.72 | 20.49 | 20.50 | 19.79 | 0.0410 | 0.0687 | 0.1091 | 0.4485 |
| | 9 | | 20.80 | 20.45 | | | 0.0809 | 0.1377 | |
| K | 0 | 20.76 | 20.76 | 20.76 | 20.76 | 0.0414 | 0.0414 | 0.0414 | 0.0414 |
| | 1 | | | 20.51 | 20.44 | | | 0.0405 | 0.0507 |
| | 2 | | | 20.77 | 21.13 | | | 0.0474 | 0.0761 |
| | 3 | 20.91 | 20.89 | 20.70 | 20.78 | 0.0424 | 0.0430 | 0.0427 | 0.0771 |
| | 6 | 20.78 | 20.85 | 20.33 | 20.73 | 0.0403 | 0.0419 | 0.0425 | 0.1492 |
| | 9 | | 20.97 | 20.99 | | | 0.0418 | 0.0574 | |
| | 12 | 20.74 | 20.85 | 20.94 | | 0.0394 | 0.0432 | 0.0667 | |
| | 18 | | 20.91 | 20.89 | | | 0.0560 | 0.0820 | |
| | 24 | | 21.25 | 21.26 | | | 0.0542 | 0.0940 | |
| L | 0 | 21.06 | 21.06 | 21.06 | 21.06 | 0.0420 | 0.0420 | 0.0420 | 0.0420 |
| | 1 | | | 20.48 | 20.71 | | | 0.0410 | 0.0426 |
| | 2 | | | 21.07 | 21.10 | | | 0.0459 | 0.0524 |
| | 3 | 20.80 | 20.90 | 21.02 | 21.14 | 0.0416 | 0.0422 | 0.0433 | 0.0608 |
| | 6 | 20.90 | 20.86 | 21.07 | 20.99 | 0.0410 | 0.0417 | 0.0429 | 0.0954 |
| | 9 | | 21.02 | 21.16 | | | 0.0424 | 0.0464 | |
| | 12 | 20.84 | 20.92 | 21.09 | | 0.0403 | 0.0443 | 0.0600 | |
| M | 0 | 20.81 | 20.81 | 20.81 | 20.81 | 0.0414 | 0.0414 | 0.0414 | 0.0414 |
| | 1 | | | 20.62 | 20.47 | | | 0.0537 | 0.1571 |
| | 2 | | | 21.07 | 20.49 | | | 0.0791 | 0.5258 |
| | 3 | 20.79 | 20.80 | 20.94 | 19.38 | 0.0415 | 0.0612 | 0.0950 | 1.8215 |
| | 6 | 20.64 | 20.67 | 20.48 | 15.47 | 0.0393 | 0.0792 | 0.2013 | 6.3998 |
| | 9 | | 20.81 | 20.45 | | | 0.1060 | 0.4246 | |
| | 12 | 20.86 | 20.68 | 19.87 | | 0.0408 | 0.1736 | 0.9025 | |
| N | 0 | 20.59 | 20.59 | 20.59 | 20.59 | 0.0585 | 0.0585 | 0.0585 | 0.0585 |
| | 1 | | | 19.82 | 18.50 | | | 1.0073 | 3.4322 |
| | 2 | | | 19.39 | 15.06 | | | 2.7643 | 8.2800 |
| | 3 | 20.59 | 19.87 | 18.52 | 13.27 | 0.1426 | 2.2486 | 4.3853 | 8.2578 |
| O | 0 | 20.63 | 20.63 | 20.63 | 20.63 | 0.0622 | 0.0622 | 0.0622 | 0.0622 |
| | 1 | | | 19.89 | 17.58 | | | 1.2470 | 5.0044 |
| | 2 | | | 19.35 | 13.50 | | | 3.2131 | 9.5896 |
| | 3 | 20.59 | 19.15 | 17.48 | 10.61 | 0.1053 | 2.8147 | 5.3725 | 8.8748 |
| P | 0 | 20.82 | 20.82 | 20.82 | 20.82 | 0.0421 | 0.0421 | 0.0421 | 0.0421 |
| | 1 | | | 20.79 | 20.86 | | | 0.0413 | 0.0414 |
| | 2 | | | 21.15 | 20.96 | | | 0.0472 | 0.0572 |
| | 3 | 20.58 | 20.84 | 20.72 | 20.56 | 0.0413 | 0.0428 | 0.0433 | 0.0680 |
| | 6 | 20.44 | 20.55 | 20.61 | 20.55 | 0.0415 | 0.0515 | 0.0463 | 0.1276 |
| | 9 | | 20.64 | 20.63 | | | 0.0674 | 0.0641 | |

TABLE 6
Stability Data - Degradation Products

| | | Degradation Product 1 (mg/mL) | | | | Degradation Product 2 (mg/mL) | | | |
|---|---|---|---|---|---|---|---|---|---|
| | | Storage Condition | | | | Storage Condition | | | |
| | Time | | 25° C./ | 30° C./ | 40° C./ | | 25° C./ | 30° C./ | 40° C./ |
| Batch | (months) | 5° C. | 60% RH | 65% RH | 75% RH | 5° C. | 60% RH | 65% RH | 75% RH |
| A | 1 | | | 0.6179 | 2.6058 | | | 0.0584 | 0.2335 |
| | 2 | | | 2.1199 | 6.4214 | | | 0.1780 | 1.1797 |
| | 3 | 0.0705 | 1.7043 | 3.6717 | 6.9159 | 0.0398 | 0.0991 | 0.3339 | 2.1930 |
| B | 1 | | | 0.7166 | 4.1588 | | | 0.0595 | 0.4136 |
| | 2 | | | 2.5557 | 6.8172 | | | 0.2453 | 1.2021 |
| | 3 | 0.0784 | 1.6254 | 4.4014 | 7.1159 | 0.0416 | 0.0938 | 0.4242 | 2.2807 |
| C | 1 | | | 0.1113 | 1.3427 | | | 0.0458 | 0.1346 |
| | 2 | | | 0.4400 | 5.0842 | | | 0.0652 | 0.9642 |
| | 3 | 0.0091 | 0.2366 | 1.1807 | 6.6767 | 0.0424 | 0.0485 | 0.0957 | 1.5515 |
| D | 1 | | | 0.0095 | 0.0560 | | | 0.0420 | 0.0454 |
| | 2 | | | 0.0233 | 0.2494 | | | 0.0489 | 0.0784 |
| | 3 | 0.0000 | 0.014 | 0.0346 | 0.8104 | 0.0441 | 0.0443 | 0.0427 | 0.1190 |
| | 6 | 0.0000 | 0.0275 | 0.2378 | 4.4255 | 0.0401 | 0.0455 | 0.0520 | 0.799 |
| | 9 | | 0.0507 | 0.2112 | | | 0.0439 | 0.0589 | |
| | 12 | 0.0000 | 0.0676 | 0.4328 | | 0.0402 | 0.0460 | 0.0570 | |
| E | 1 | | | 0.0514 | 0.0616 | | | 0.0442 | 0.0481 |
| | 2 | | | 0.0261 | 0.3021 | | | 0.0491 | 0.0832 |
| | 3 | 0.0000 | 0.1025 | 0.0543 | 0.9089 | 0.0416 | 0.0451 | 0.0470 | 0.1255 |
| F | 1 | | | 0.2238 | 1.8855 | | | 0.0513 | 0.1134 |
| | 2 | | | 1.0365 | 4.7978 | | | 0.0957 | 0.8818 |
| | 3 | 0.0254 | 0.5596 | 2.2277 | 6.7866 | 0.0414 | 0.0596 | 0.1437 | 1.4433 |
| G | 1 | | | 0.0137 | 0.1112 | | | 0.0426 | 0.0424 |
| | 2 | | | 0.0322 | 0.4781 | | | 0.0476 | 0.1039 |
| | 3 | 0.0000 | 0.0196 | 0.0515 | 1.8262 | 0.0433 | 0.0433 | 0.0497 | 0.2055 |
| | 6 | 0.0000 | 0.0364 | 0.1351 | 5.6712 | 0.0408 | 0.0461 | 0.0474 | 0.7449 |
| | 9 | | 0.0595 | 0.3515 | | | 0.0450 | 0.0669 | |
| | 12 | 0.0000 | 0.0984 | 0.9122 | | 0.0398 | 0.0457 | 0.0591 | |
| H | 1 | | | 0.0000 | 0.0128 | | | 0.0427 | 0.0467 |
| | 2 | | | 0.0000 | 0.0317 | | | 0.0477 | 0.0561 |
| | 3 | 0.0000 | 0.0000 | 0.0000 | 0.0591 | 0.0415 | 0.0421 | 0.0436 | 0.0453 |
| | 6 | 0.0000 | 0.0000 | 0.0116 | 0.1725 | 0.0404 | 0.0426 | 0.0418 | 0.0719 |
| | 9 | | 0.0000 | 0.0172 | | | 0.0420 | 0.0463 | |
| | 12 | 0.0000 | 0.0076 | 0.0207 | | 0.0396 | 0.0440 | 0.0500 | |
| I | 1 | | | 0.0185 | 0.0706 | | | 0.0403 | 0.0503 |
| | 2 | | | 0.0315 | 0.1446 | | | 0.0496 | 0.0717 |
| | 3 | 0.0000 | 0.0213 | 0.0000 | 0.2149 | 0.0419 | 0.0436 | 0.0483 | 0.0612 |
| | 6 | 0.0000 | 0.0333 | 0.0723 | 0.5021 | 0.0405 | 0.0454 | 0.0459 | 0.1078 |
| | 9 | | 0.0437 | 0.1057 | | | 0.0445 | 0.0527 | |
| | 12 | 0.0000 | 0.0600 | 0.1548 | | 0.0397 | 0.0446 | 0.0541 | |
| J | 1 | | | 0.0147 | 0.0568 | | | 0.0410 | 0.0466 |
| | 2 | | | 0.0265 | 0.1168 | | | 0.0492 | 0.0681 |
| | 3 | 0.0000 | 0.0165 | 0.0345 | 0.1781 | 0.0431 | 0.0445 | 0.0480 | 0.0932 |
| | 6 | 0.0000 | 0.0268 | 0.0606 | 0.3371 | 0.0410 | 0.0419 | 0.0485 | 0.1114 |
| | 9 | | 0.0353 | 0.0810 | | | 0.0456 | 0.0567 | |
| K | 1 | | | 0.0000 | 0.0085 | | | 0.0405 | 0.0422 |
| | 2 | | | 0.0000 | 0.0216 | | | 0.0474 | 0.0545 |
| | 3 | 0.0000 | 0.0000 | 0.0000 | 0.0309 | 0.0424 | 0.0430 | 0.0427 | 0.0462 |
| | 6 | 0.0000 | 0.0000 | 0.0000 | 0.0815 | 0.0403 | 0.0419 | 0.0425 | 0.0677 |
| | 9 | | 0.0000 | 0.0113 | | | 0.0418 | 0.0461 | |
| | 12 | 0.0000 | 0.0000 | 0.0169 | | 0.0394 | 0.0432 | 0.0498 | |
| | 18 | | 0.0091 | 0.0211 | | | 0.0469 | 0.0609 | |
| | 24 | | 0.0098 | 0.0362 | | | 0.0444 | 0.0578 | |
| L | 1 | | | 0.0000 | 0.0000 | | | 0.0410 | 0.0426 |
| | 2 | | | 0.0000 | 0.0000 | | | 0.0459 | 0.0524 |
| | 3 | 0.0000 | 0.0000 | 0.0000 | 0.0141 | 0.0416 | 0.0422 | 0.0433 | 0.0467 |
| | 6 | 0.0000 | 0.0000 | 0.0000 | 0.0300 | 0.0410 | 0.0417 | 0.0429 | 0.0654 |
| | 9 | | 0.0000 | 0.0000 | | | 0.0424 | 0.0464 | |
| | 12 | 0.0000 | 0.0000 | 0.0100 | | 0.0403 | 0.0443 | 0.0500 | |
| M | 1 | | | 0.0129 | 0.1048 | | | 0.0408 | 0.0523 |
| | 2 | | | 0.0304 | 0.4309 | | | 0.0487 | 0.0949 |
| | 3 | 0.0000 | 0.0178 | 0.0502 | 1.6359 | 0.0415 | 0.0434 | 0.0448 | 0.1856 |
| | 6 | 0.0000 | 0.0358 | 0.1485 | 5.4817 | 0.0393 | 0.0434 | 0.0528 | 0.9181 |
| | 9 | | 0.0600 | 0.3548 | | | 0.0460 | 0.0698 | |
| | 12 | 0.0000 | 0.1249 | 0.8406 | | 0.0408 | 0.0487 | 0.0619 | |
| N | 1 | | | 0.9345 | 3.1489 | | | 0.0728 | 0.2833 |
| | 2 | | | 2.5687 | 6.5827 | | | 0.1956 | 1.6973 |
| | 3 | 0.1001 | 2.1300 | 4.0528 | 7.0330 | 0.0425 | 0.1186 | 0.3325 | 1.2248 |
| O | 1 | | | 1.1669 | 4.4406 | | | 0.0801 | 0.5638 |
| | 2 | | | 2.9423 | 7.4382 | | | 0.2708 | 2.1514 |
| | 3 | 0.0644 | 2.6836 | 4.8970 | 6.4493 | 0.0409 | 0.1311 | 0.4755 | 2.4255 |
| P | 1 | | | 0.0000 | 0.0000 | | | 0.0413 | 0.0414 |
| | 2 | | | 0.0000 | 0.0000 | | | 0.0472 | 0.0572 |
| | 3 | 0.0000 | 0.0000 | 0.0000 | 0.0127 | 0.0413 | 0.0428 | 0.0433 | 0.0553 |
| | 6 | 0.0000 | 0.0089 | 0.0000 | 0.0384 | 0.0415 | 0.0426 | 0.0463 | 0.0892 |
| | 9 | | 0.0222 | 0.0119 | | | 0.0452 | 0.0522 | |

TABLE 7
Stability Data -- BHA and BHT. Shaded cells indicate that data was not collected at that
time point and condition for the analytical property. Note that Formulations A, B, N,
and O, which did not contain any BHA or BHA, were not evaluated for these properties
and are not listed in this table. Some formulations contained only one of BHA or BHT.

| | | BHA (mg/mL) | | | | BHT (mg/mL) | | | |
|---|---|---|---|---|---|---|---|---|---|
| | | Storage Condition | | | | Storage Condition | | | |
| | Time | | 25° C./ | 30° C./ | 40° C./ | | 25° C./ | 30° C./ | 40° C./ |
| Batch | (months) | 5° C. | 60% RH | 65% RH | 75% RH | 5° C. | 60% RH | 65% RH | 75% RH |
| C | 0 | | | | | 0.1695 | 0.1695 | 0.1695 | 0.1695 |
| | 1 | | | | | | | 0.1106 | 0.0078 |
| | 2 | | | | | | | 0.0342 | 0.0073 |
| | 3 | | | | | 0.1586 | 0.0733 | 0.0110 | 0.0117 |
| D | 0 | | | | | 0.7472 | 0.7472 | 0.7472 | 0.7472 |
| | 1 | | | | | | | 0.6999 | 0.5220 |
| | 2 | | | | | | | 0.6290 | 0.2373 |
| | 3 | | | | | 0.7370 | 0.6541 | 0.5795 | 0.0444 |
| | 6 | | | | | 0.7157 | 0.5852 | 0.3630 | 0.0082 |
| | 9 | | | | | | 0.5005 | 0.2335 | |
| | 12 | | | | | 0.7265 | 0.4687 | 0.1155 | |
| E | 0 | | | | | 0.7313 | 0.7313 | 0.7313 | 0.7313 |
| | 1 | | | | | | | 0.6190 | 0.5238 |
| | 2 | | | | | | | 0.6225 | 0.2270 |
| | 3 | | | | | 0.6820 | 0.4952 | 0.5132 | 0.0417 |
| | 6 | | | | | | | | |
| | 9 | | | | | | | | |
| F | 0 | 0.0382 | 0.0382 | 0.0382 | 0.0382 | | | | |
| | 1 | | | 0.0162 | 0.0088 | | | | |
| | 2 | | | 0.0075 | 0.0234 | | | | |
| | 3 | 0.0365 | 0.0049 | 0.0000 | 0.0196 | | | | |
| | 6 | | | | | | | | |
| | 9 | | | | | | | | |
| G | 0 | 0.0392 | 0.0392 | 0.0392 | 0.0392 | 0.4226 | 0.4226 | 0.4226 | 0.4226 |
| | 1 | | | 0.0383 | 0.0255 | | | 0.3839 | 0.2248 |
| | 2 | | | 0.0361 | 0.0172 | | | 0.3292 | 0.0451 |
| | 3 | 0.0400 | 0.0377 | 0.0337 | 0.0000 | 0.4136 | 0.3559 | 0.2849 | 0.0086 |
| | 6 | 0.0395 | 0.0353 | 0.0236 | 0.0000 | 0.3960 | 0.3001 | 0.1629 | 0.0081 |
| | 9 | | 0.0320 | 0.0089 | | | 0.2526 | 0.0529 | |
| | 12 | 0.0389 | 0.0281 | 0.0000 | | 0.4098 | 0.2021 | 0.0146 | |
| H | 0 | 0.1611 | 0.1611 | 0.1611 | 0.1611 | 0.7403 | 0.7403 | 0.7403 | 0.7403 |
| | 1 | | | 0.1591 | 0.1539 | | | 0.7144 | 0.6235 |
| | 2 | | | 0.1606 | 0.1454 | | | 0.6790 | 0.4907 |
| | 3 | 0.1616 | 0.1600 | 0.1577 | 0.1277 | 0.7338 | 0.6980 | 0.6622 | 0.3407 |
| | 6 | 0.1598 | 0.1598 | 0.1572 | 0.0820 | 0.7058 | 0.6613 | 0.6165 | 0.1214 |
| | 9• | | 0.1592 | 0.1547 | | | 0.6499 | 0.5600 | |
| | 12 | 0.1580 | 0.1571 | 0.1490 | | 0.7248 | 0.6244 | 0.5151 | |
| I | 0 | 0.4054 | 0.4054 | 0.4054 | 0.4054 | | | | |
| | 1 | | | 0.3721 | 0.2864 | | | | |
| | 2 | | | 0.3599 | 0.2221 | | | | |
| | 3 | 0.4024 | 0.3708 | 0.3385 | 0.1615 | | | | |
| | 6 | 0.3997 | 0.3528 | 0.2967 | 0.0503 | | | | |
| | 9 | | 0.3320 | 0.2488 | | | | | |
| | 12 | 0.4019 | 0.3161 | 0.2173 | | | | | |
| J | 0 | 0.4023 | 0.4023 | 0.4023 | 0.4023 | | | | |
| | 1 | | | 0.3767 | 0.3039 | | | | |
| | 2 | | | 0.3637 | 0.2507 | | | | |
| | 3 | 0.4028 | 0.3810 | 0.3507 | 0.1924 | | | | |
| | 6 | 0.4001 | 0.3584 | 0.3108 | 0.1090 | | | | |
| | 9 | | 0.3423 | 0.2816 | | | | | |
| K | 0 | 0.4123 | 0.4123 | 0.4123 | 0.4123 | 0.4202 | 0.4202 | 0.4202 | 0.4202 |
| | 1 | | | 0.4108 | 0.3921 | | | 0.3914 | 0.3271 |
| | 2 | | | 0.4177 | 0.3932 | | | 0.3775 | 0.2795 |
| | 3 | 0.4158 | 0.4142 | 0.4102 | 0.3704 | 0.4121 | 0.3911 | 0.3583 | 0.2122 |
| | 6 | 0.4150 | 0.4113 | 0.4081 | 0.3050 | 0.4043 | 0.3705 | 0.3365 | 0.0849 |
| | 9 | | 0.4138 | 0.4055 | | | 0.3651 | 0.3150 | |
| | 12 | 0.4124 | 0.4074 | 0.3963 | | 0.3933 | 0.3335 | 0.2688 | |
| | 18 | | 0.4093 | 0.3890 | | | 0.3160 | 0.2181 | |
| | 24 | | 0.4111 | 0.3819 | | | 0.2173 | 0.1908 | |
| L | 0 | 0.4129 | 0.4129 | 0.4129 | 0.4129 | 0.7492 | 0.7492 | 0.7492 | 0.7492 |
| | 1 | | | 0.4082 | 0.3986 | | | 0.7019 | 0.6630 |
| | 2 | | | 0.4186 | 0.4095 | | | 0.7225 | 0.6122 |
| | 3 | 0.4119 | 0.4115 | 0.4121 | 0.3976 | 0.7227 | 0.7018 | 0.6966 | 0.5300 |
| | 6 | 0.4117 | 0.4121 | 0.4165 | 0.3809 | 0.7353 | 0.6966 | 0.6699 | 0.4048 |
| | 9 | | 0.4130 | 0.4110 | | | 0.6783 | 0.6261 | |
| | 12 | 0.4117 | 0.4099 | 0.4061 | | 0.7233 | 0.6514 | 0.5679 | |
| M | 0 | 0.0419 | 0.0419 | 0.0419 | 0.0419 | 0.4186 | 0.4186 | 0.4186 | 0.4186 |
| | 1 | | | 0.0381 | 0.0232 | | | 0.3642 | 0.2095 |
| | 2 | | | 0.0400 | 0.0170 | | | 0.3302 | 0.0428 |
| | 3 | 0.0422 | 0.0400 | 0.0356 | 0.0000 | 0.4031 | 0.3517 | 0.2751 | 0.0087 |
| | 6 | 0.0427 | 0.0383 | 0.0248 | 0.0000 | 0.3979 | 0.3053 | 0.1466 | 0.0064 |
| | 9 | | 0.0340 | 0.0207 | | | 0.2419 | 0.0478 | |
| | 12 | 0.0422 | 0.0280 | 0.0000 | | 0.3809 | 0.1663 | 0.0132 | |
| P | 0 | 0.4046 | 0.4046 | 0.4046 | 0.4046 | 0.7549 | 0.7549 | 0.7549 | 0.7549 |
| | 1 | | | 0.3997 | 0.3932 | | | 0.7157 | 0.6674 |
| | 2 | | | 0.4098 | 0.3995 | | | 0.7132 | 0.6214 |
| | 3 | 0.4001 | 0.4026 | 0.397 | 0.3855 | 0.7263 | 0.6922 | 0.6462 | 0.5639 |
| | 6 | 0.4036 | 0.4047 | 0.3992 | 0.3585 | 0.7103 | 0.6362 | 0.6310 | 0.3776 |
| | 9 | | 0.3853 | 0.3883 | | | 0.5296 | 0.5593 | |

TABLE 8
Stability Data - Tube Weights

| | | Tube Weights (Weight of Ten Tubes) (g) | | | |
|---|---|---|---|---|---|
| | Time | Storage Condition | | | |
| Batch | (months) | 5° C. | 25° C./60% RH | 30° C./65% RH | 40° C./75% RH |
| A | 0 | 14.7513 | 14.7060 | 14.6179 | 14.6768 |
| | 1 | 14.7584 | 14.7077 | 14.6187 | 14.6674 |
| | 2 | 14.7659 | 14.7071 | 14.6147 | 14.6584 |
| | 3 | 14.7715 | 14.7062 | 14.6114 | 14.6507 |
| B | 0 | 14.4666 | 14.4899 | 14.4725 | 14.5510 |
| | 1 | 14.4751 | 14.4949 | 14.4853 | 14.5681 |
| | 2 | 14.4818 | 14.4999 | 14.4880 | 14.5755 |
| | 3 | 14.4854 | 14.5011 | 14.4891 | 14.5760 |
| C | 0 | 14.6277 | 14.6706 | 14.6776 | 14.6817 |
| | 1 | 14.6359 | 14.6692 | 14.6794 | 14.6762 |
| | 2 | 14.6465 | 14.6721 | 14.6758 | 14.6675 |
| | 3 | 14.6472 | 14.6696 | 14.6722 | 14.6564 |
| D | 0 | 14.7208 | 14.6966 | 14.6674 | 14.7390 |
| | 1 | 14.7255 | 14.6960 | 14.6664 | 14.7314 |
| | 2 | 14.7363 | 14.6995 | 14.6634 | 14.7224 |
| | 3 | 14.7398 | 14.6977 | 14.6598 | 14.7117 |
| | 6 | 14.7416 | 14.6893 | 14.6417 | 14.6523 |
| | 9 | 14.7268 | 14.6797 | 14.6329 | |
| E | 0 | 14.4838 | 14.4542 | 14.4438 | 14.4623 |
| | 1 | 14.4901 | 14.4617 | 14.4541 | 14.4794 |
| | 2 | 14.5052 | 14.4663 | 14.4583 | 14.4858 |
| | 3 | 14.5062 | 14.4674 | 14.4588 | 14.4852 |
| F | 0 | 14.7466 | 14.7266 | 14.6074 | 14.5979 |
| | 1 | 14.75.36 | 14.7302 | 14.6170 | 14.5898 |
| | 2 | 14.7636 | 14.7279 | 14.6145 | 14.5834 |
| | 3 | 14.7654 | 14.7272 | 14.6099 | 14.5717 |
| G | 0 | 14.7058 | 14.6671 | 14.6821 | 14.7006 |
| | 1 | 14.7117 | 14.6687 | 14.6815 | 14.6924 |
| | 2 | 14.7221 | 14.6672 | 14.6794 | 14.6824 |
| | 3 | 14.7237 | 14.6658 | 14.6758 | 14.6731 |
| | 6 | 14.7245 | 14.6573 | 14.6595 | 14.6223 |
| | 9 | 14.7137 | 14.6495 | 14.6471 | |
| | 12 | 14.7239 | 14.6433 | 14.6383 | |
| H | 0 | 14.722 | 14.6787 | 14.7400 | 14.7321 |
| | 1 | 14.7253 | 14.6794 | 14.7398 | 14.7268 |
| | 2 | 14.7358 | 14.6787 | 14.7386 | 14.7154 |
| | 3 | 14.7390 | 14.6756 | 14.7343 | 14.7067 |
| | 6 | 14.7412 | 14.6677 | 14.7168 | 14.6516 |
| | 9 | 14.7247 | 14.6596 | 14.7056 | |
| | 12 | 14.7391 | 14.6535 | 14.6967 | |
| I | 0 | 14.7014 | 14.7192 | 14.7015 | 14.6962 |
| | 1 | 14.7044 | 14.7191 | 14.7004 | 14.6909 |
| | 2 | 14.7178 | 14.7169 | 14.699 | 14.6784 |
| | 3 | 14.7187 | 14.7154 | 14.6955 | 14.6705 |
| | 6 | 14.7199 | 14.7069 | 14.6782 | 14.6179 |
| | 9 | 14.7089 | 14.6995 | 14.6669 | |
| | 12 | 14.7185 | 14.6939 | 14.6558 | |
| J | 0 | 14.5882 | 14.4953 | 14.4239 | 14.4065 |
| | 1 | 14.5957 | 14.5005 | 14.4328 | 14.4243 |
| | 2 | 14.6036 | 14.5052 | 14.4373 | 14.4267 |
| | 3 | 14.6077 | 14.5063 | 14.4393 | 14.4292 |
| | 6 | 14.6105 | 14.5165 | 14.4509 | 14.4335 |
| | 9 | 14.5994 | 14.5174 | 14.4518 | |
| K | 0 | 14.7364 | 14.6732 | 14.6724 | 14.5602 |
| | 1 | 14.7436 | 14.6731 | 14.6716 | 14.5532 |
| | 2 | 14.7548 | 14.6723 | 14.6702 | 14.5414 |
| | 3 | 14.758 | 14.6703 | 14.6668 | 14.5334 |
| | 6 | 14.7592 | 14.6613 | 14.6492 | 14.4778 |
| | 9 | 14.7490 | 14.6536 | 14.6371 | |
| | 12 | 14.7578 | 14.6471 | 14.6280 | |
| | 18 | | 14.6332 | 14.6011 | |
| | 24 | | 14.6133 | 14.5697 | |
| L | 0 | 14.6653 | 14.6835 | 14.6593 | 14.6733 |
| | 1 | 14.6717 | 14.6818 | 14.6589 | 14.6685 |
| | 2 | 14.6797 | 14.6835 | 14.6565 | 14.6594 |
| | 3 | 14.6836 | 14.6832 | 14.6528 | 14.6488 |
| | 6 | 14.6856 | 14.6727 | 14.6359 | 14.5936 |
| | 9 | 14.671 | 14.6649 | 14.6223 | |
| | 12 | 14.683 | 14.6603 | 14.6141 | |
| M | 0 | 14.7147 | 14.6836 | 14.5370 | 14.7597 |
| | 1 | 14.7126 | 14.6793 | 14.5325 | 14.7492 |
| | 2 | 14.7222 | 14.6786 | 14.5309 | 14.7393 |
| | 3 | 14.7256 | 14.6775 | 14.5284 | 14.7329 |
| | 6 | 14.7263 | 14.6685 | 14.5107 | 14.6778 |
| | 9 | 14.7184 | 14.6609 | 14.4986 | |
| | 12 | 14.7242 | 14.6558 | 14.4905 | |
| N | 0 | 14.6468 | 14.7351 | 14.7585 | 14.6898 |
| | 1 | 14.6542 | 14.7308 | 14.755 | 14.6820 |
| | 2 | 14.6643 | 14.7308 | 14.753 | 14.6723 |
| | 3 | 14.6667 | 14.7292 | 14.7501 | 14.6645 |
| O | 0 | 14.5036 | 14.5400 | 14.5263 | 14.5017 |
| | 1 | 14.5086 | 14.5408 | 14.5334 | 14.5152 |
| | 2 | 14.5166 | 14.5490 | 14.539 | 14.5214 |
| | 3 | 14.5177 | 14.5498 | 14.5402 | 14.5235 |
| P | 0 | 14.4546 | 14.2812 | 14.5182 | 14.3715 |
| | 1 | 14.4595 | 14.2813 | 14.5261 | 14.3890 |
| | 2 | 14.468 | 14.2894 | 14.5307 | 14.3909 |
| | 3 | 14.4691 | 14.2907 | 14.5307 | 14.3939 |
| | 6 | 14.4749 | 14.3016 | 14.5448 | 14.3968 |
| | 9 | 14.4674 | 14.3026 | 14.5458 | |

### Long Term Stability Analysis of Targeted Formulation

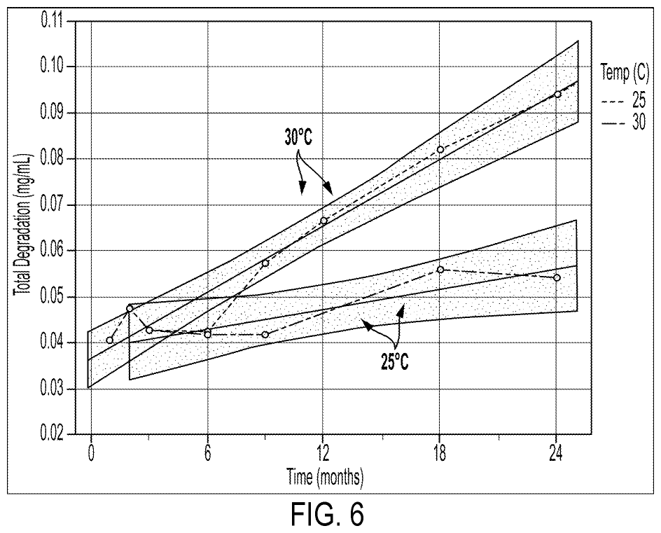

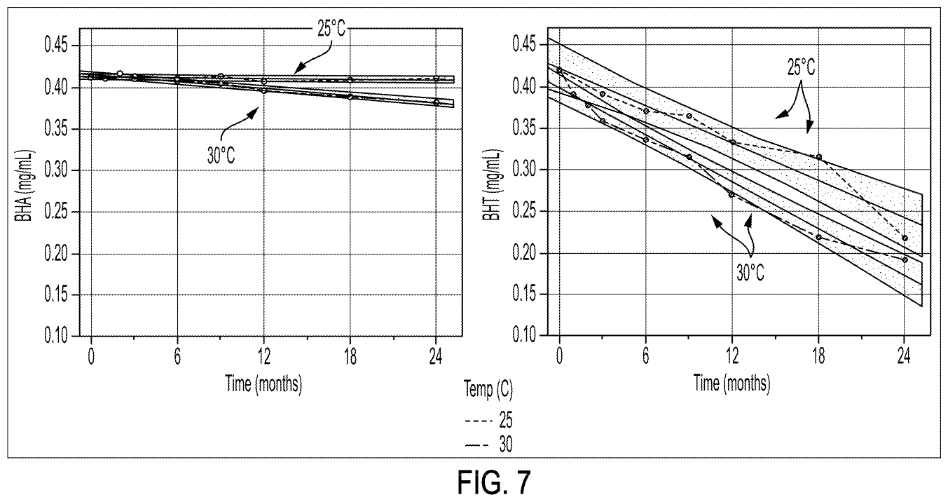

Data through 24 months for formulation K from Tables 5-8 is shown in FIGS. 6-8. This portion of the analysis focuses on this formulation (Formulation Batch K, contains 0.5%/0.5% wt/wt of BHA/BHT). An upward trend in the potency results was observed which was unexpected, but the results for the total degradation products are well within an acceptable range for good product quality at both 25° C. and 30° C. (FIG. 6). BHT is preferentially oxidized and is consumed more rapidly than BHA, but the total amount of BHA and BHT remains well above 10% of the starting BHA and BHT levels (FIG. 7).

To further understand potential causes for the observed increase in potency, the tube weight data was examined (see Table 8). This data shows trends in the weights that are correlated with the increase in potency. In particular, the weight trends show that there is some evidence of weight loss for the PF113 tubes and also weight gain for the PF413 tubes.

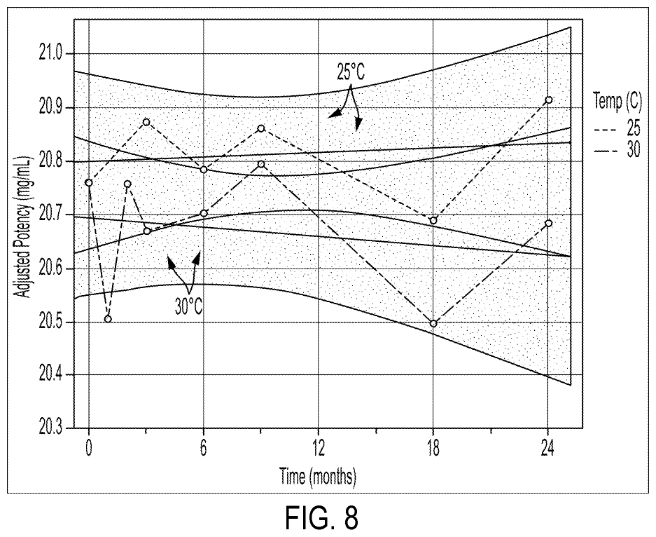

No noticeable liquid was observed leaking from PF113 tubes. All of the tubes were filled and sealed manually for this study, and one potential cause of the downward trending for the PF113 package material is that the tube cap did not have a tight or lasting seal and allowed the formulation to slowly lose volume and mass due to evaporation of ethanol vapor. Another possibility is that the enthanol solvent leaked from the sealed end of the tube due to a manual non-optimized seal. Assuming that the loss in weight for Formulation batch K is due to evaporative loss of ethanol, an adjusted potency (corrected for volume of EtOH loss) is shown in FIG. 8. There is no significant trend in the adjusted potency results.

The starting potency is noticeably above the target potency of 20 mg/mL, which makes the adjusted results appear to be very close to the current approved upper regulatory acceptance limit of 21.0 mg/mL. This may have been due to the manual filling for these formulation batches, where some evaporative loss of ethanol (EtOH) may have occurred prior to filling into the tubes, and commercial production of this material will used automated filling lines under more controlled conditions.

The potential improvement in stability due to filling in a nitrogen atmosphere and the differences between the two tube types were also assessed, but neither were found to be significant factors that affect stability.

### Discussion of Results

A summary of relevant data from Table 5 is shown below in Table 9. All formulations in Table 9 did not use a nitrogen blanket during filling and used tube type PF113. In Table 9, “0” indicates 0 mg/mL of BHA and/or BHT, “L” indicates 0.0404 mg/mL for BHA and 0.162 mg/mL for BHT, “M” indicates 0.162 mg/mL for BHA and 0.404 mg/mL for BHT, “H” indicates 0.404 mg/mL for BHA and 0.727 mg/mL for BHT. The total active (buprenorphine hydrochloride) degradation product values reported in Table 9 were calculated by subtracting the initial (0 months) total degradation products from the measured total degradation products at 3 or 12 months at the given storage temperature (25° C., 30° C., or 40° C.) and relative humidity from Table 5.

TABLE 9
BHA and BHT reduce total buprenorphine hydrochloride degradation products for extended
time periods and under room temperature or elevated temperature storage conditions

| | | | 12 months @ | 3 months @ | 3 months @ | 3 months @, |
|---|---|---|---|---|---|---|
| Letter | | | 25° C./60% RH | 25° C./60% RH | 30° C./65% RH | 40° C./75% RH |
| Code | BHA | BHT | (mg/mL) | (mg/mL) | (mg/mL) | (mg/mL) |
| D | 0 | H | 0.0714 | 0.0161 | 0.0351 | 0.8872 |
| G | L | M | 0.1029 | 0.0217 | 0.06 | 1.9905 |
| H | M | H | 0.0105 | 0.001 | 0.0025 | 0.0633 |
| I | H | 0 | 0.063 | 0.0233 | 0.0505 | 0.2345 |
| K | H | M | 0.0018 | 0.0016 | 0.0013 | 0.0357 |
| L | H | H | 0.0023 | 0.0002 | 0.0013 | 0.0188 |
| M | L | M | 0.1322 | 0.0198 | 0.0536 | 1.7801 |
| N | 0 | 0 | — | 2.1901 | 4.3268 | 8.1993 |

When the tubes were stored for 12 months at 25° C. and at 60% relative humidity, total buprenorphine hydrochloride degradation products of test formulations containing BHA alone (Formulation I), BHT alone (Formulation D) or both BHA and BHT (Formulations G, H, K, L and M) ranged from 0.0018 to 0.1322 mg/mL. In contrast, Formulation N (containing no BHA or BHT) contained 2.1901 mg/mL of total degradation products just after 3 months at 25° C. Thus, at room temperature, formulations containing at least one of BHA or BHT unexpectedly exhibited significantly lower concentrations of degradation products (i.e., exhibited greater buprenorphine hydrochloride stability) even after a year of storage as compared to Formulation N without BHA or BHT after only a quarter of the storage time (3 months) at room temperature.

This surprising improvement in formulation stability was also observed at higher storage temperatures and relative humidities (both of which can negatively affect stability). After 3 months at 30° C. and 65% relative humidity, the concentration of total buprenorphine hydrochloride degradation products of test Formulations D, G, H, I, K, L and M containing at least one of BHA and BHT ranged from 0.0013 to 0.06 mg/mL. In contrast, Formulation N contained 4.3268 mg/mL total buprenorphine hydrochloride degradation products. Similarly, after 3 months at 40° C. ad 75% relative humidity, the concentration of total degradation products of test Formulations D, G, H, I, K, L and M ranged from 0.0188 to 1.9905 mg/mL, while Formulation N contained 8.1993 mg/mL total degradation products. Thus, after 3 months at an elevated storage temperature of 30° C. or 40° C., formulations with at least one of BHA or BHT surprisingly exhibited significantly lower concentrations of buprenorphine hydrochloride degradation products as compared to Formulation N without BHA or BHT.

Further, formulations containing high (H) or medium (M) levels of both BHA and BHT (Formulations H, K, and L) were even more stable as compared to formulations containing only one of BHA and BHT (Formulations D and I) and formulations with low (L) levels of BHA or BHT (Formulations G and M). Formulations H, K, and L had the lowest accumulated total degradation products after 12 months at 25° C. and 3 months at 25° C., 30° C., and 40° C.

The BHA/BHT stability data in Table 7 further emphasize these unexpected results. For example, after 12 months at 25° C., the remaining concentration of BHA is lower for Formulation I containing no BHT (77.97% of original BHA) as compared to the remaining concentration of BHA for Formulation L containing a high (H) amount of BHT (99.27% of original BHA). Similarly, after 12 months at 25° C., the remaining concentration of BHT is lower for Formulation D containing no BHA (62.73% of original BHT) as compared to the remaining concentration of BHT for Formulation L containing a high (H) amount of BHA (86.95%). Examining Formulation K containing high (H) levels of BHA and medium (M) levels of BHT after 12 months at 25° C., BHT levels (79.37% of original BHT) are further reduced as compared to BHA levels (98.81% of original BHA). Similarly, examining Formulation L containing high (H) levels of both BHA and BHT after 12 months at 25° C., BHT levels (86.95% of original BHT) are further reduced than BHA levels (99.27% of original BHA). Thus, BHT levels generally decrease at a higher rate than BHA levels, but the presence of both BHA and BHT slows the rate of decrease of both of these compounds and provides for exceptional formulation stability.

In sum, these results show that relative low concentrations of additives BHA and/or BHT, and even more preferably BHA and BHT, unexpectedly provide for greatly enhanced long term storage stability. Even more so, the results show that enhanced long term storage stability can be achieved without refrigeration even if the formulation is stored at elevated temperatures and relative humidities.

### Example 2

### 36-Month Stability Analysis

The design of this study provided for evaluating and modeling of the rate of change over time as a function of temperature. The Arrhenius Time-Scaled Least Squares (ATLS) methodology was applied to this data to estimate the expected rate of change at the desired commercial storage condition (reference temperate=25° C.) and the projected total change over the desired commercial shelf life (36 months). See Rauk et al., “Arrhenius Time-Scaled Least Squares: A Simple, Robust Approach to Accelerated Stability Data Analysis for Bioproducts,” Journal of Pharmaceutical Sciences, 103:2278-2286, 2014, DOI 10.1002/jps.24063. The model used to fit the data in this analysis is either a linear or quadratic trend model:

$$\\ \begin{matrix}{Y}_{batch} ( {t}_{ref} ) = {θ}_{batch} + {α}_{batch} {t}_{ref} or & ( 1 )\end{matrix}$$

$$\\ \begin{matrix}{Y}_{batch} ( {t}_{ref} ) = {θ}_{batch} + {α}_{batch} {t}_{ref} + {β}_{batch} {t}_{ref}^{{}_{} 2} where & ( 2 )\end{matrix}$$

$$\\ {t}_{ref} = t exp \{ \frac{- {E}_{a}}{R} ( \frac{1}{T} - \frac{1}{{T}_{ref}} ) \} and {T}_{ref} = 298.15 K ( equivalent to 25 ° C . ) .$$

This model captures the effect of the acceleration of the rate of change as temperature increases by modifying the time scale, t_{reƒ}, using the Arrhenius rate model. The model which fits the data the best (either linear (1) or quadratic (2) in Arrhenius time-scale) is chosen. The key parameter estimated in the model is the activation energy, E_{α}, and this is chosen by finding the value that minimizes the sum of squared errors for the model predictions. Each batch has an estimated intercept, θ_{batch}, a linear slope, α_{batch}, and if the quadratic model is chosen, a curvature term β_{batch}. This model allows for making predictions of the change over time for each batch at a chosen reference temperature and at given storage time. Calculations were performed using the JMP Accelerated Stability Analysis Tool (Elanco R&D Version 1.0) running in JMP Version 14.1.0.

The predicted change in potency over 36 months at 25° C. storage conditions was determined for each formulation batch (see Table 10 below). Formulation batches that showed a large amount of predicted change (>15%) were identified (formulation batches F, C, N, A, B, and O). As a result of this initial screening analysis, formulation batches F, C, N, A, B, and O were removed from further analysis on stability.

TABLE 10
Arrhenius Time-Scaled Stability Regression Model
Predicted Change in Potency at 25° C. over
36 months based on 3-month accelerated stability data

| Batch | Change in Potency (mg/mL) |
|---|---|
| A | 12.3 |
| B | 14.8 |
| C | 9.7 |
| D | 1.1 |
| E | 2.1 |
| F | 9.5 |
| G | 2.2 |
| H | −0.1 |
| I | 0.6 |
| J | 1.0 |
| K | −0.1 |
| L | −0.4 |
| M | 1.7 |
| N | 12.1 |
| O | 16.2 |
| P | 0.1 |

The predicted quality attributes from the ATLS accelerated predictive stability modeling based on 9 of months of accelerated stability data is shown in the table below. The reference temperature was set to be 25° C., and the target storage duration was chosen to be 36 months.

TABLE 11
Arrhenius Time-Scaled Stability Regression Model Predictions
(based on 9 months accelerated stability)

| | Predicted | Predicted | Predicted | Predicted |
|---|---|---|---|---|
| | BHA | BHT | Potency | Total Deg |
| | (mg/mL) | (mg/mL) | (mg/mL) | (mg/mL) |
| Batch | @ 36 months | @ 36 months | @ 36 months | @ 36 Months |
| A | 0.00 | 0.00 | 11.67 | 17.06 |
| B | 0.00 | 0.00 | 9.77 | 18.23 |
| C | 0.00 | 0.00 | 13.72 | 14.28 |
| D | 0.00 | 0.00 | 19.05 | 3.27 |
| E | 0.00 | 0.00 | 19.25 | 1.45 |
| F | 0.01 | 0.00 | 13.89 | 14.30 |
| G | 0.01 | 0.00 | 18.41 | 4.26 |
| H | 0.12 | 0.27 | 20.68 | 0.18 |
| I | 0.15 | 0.00 | 20.26 | 0.48 |
| J | 0.19 | 0.00 | 20.17 | 0.38 |
| K | 0.36 | 0.16 | 20.82 | 0.12 |
| L | 0.40 | 0.49 | 21.04 | 0.08 |
| M | 0.01 | 0.00 | 18.46 | 4.20 |
| N | 0.00 | 0.00 | 11.87 | 16.71 |
| O | 0.00 | 0.00 | 8.85 | 18.84 |
| P | 0.38 | 0.46 | 20.65 | 0.11 |

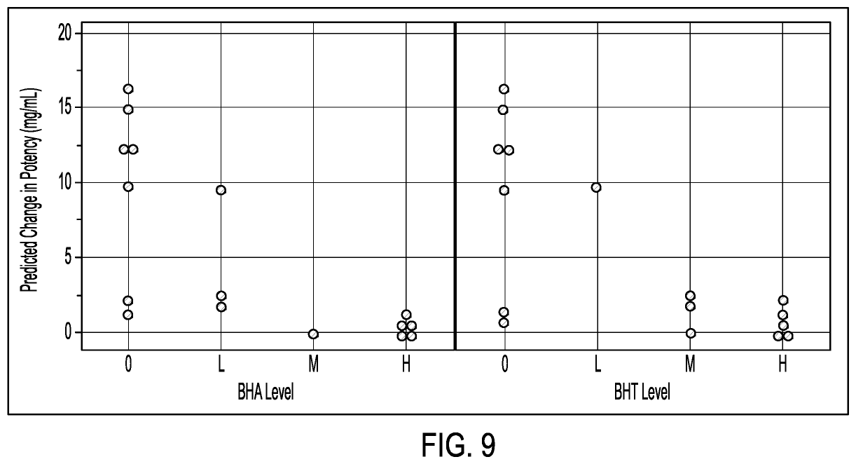

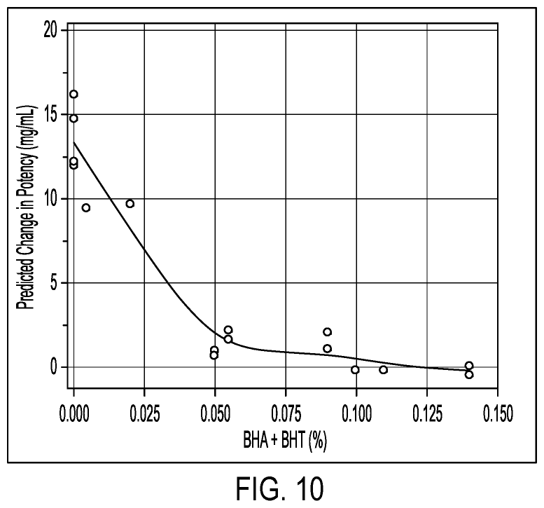

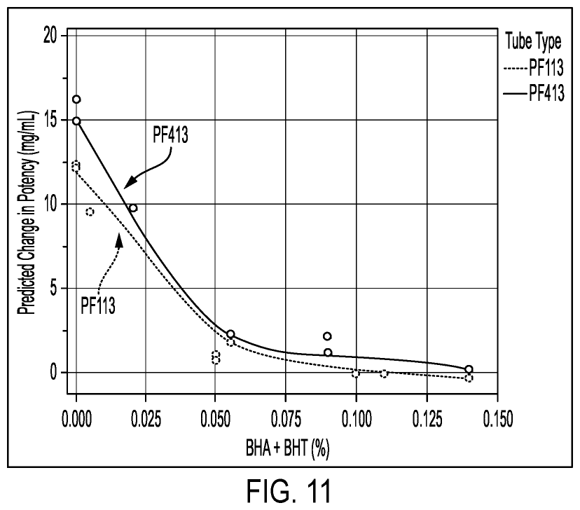

Plotting the predicted change over 36 months at 25° C. versus the coded levels for the amounts of BHA and BHT used in each formulation batch (FIG. 9) shows a strong relationship in the amount of additive used. Plotting the predicted change versus the total amount of BHA and BHT (wt/wt %) in the formulation (FIG. 10) also shows a strong relationship, and a combined amount of at least 0.05% wt/wt for the additives leads to very small, predicted change over time. Separate plots of this relationship for each tube type are shown in FIG. 11, and this indicates that there is very little difference in the rates of change due to the type of tube used.

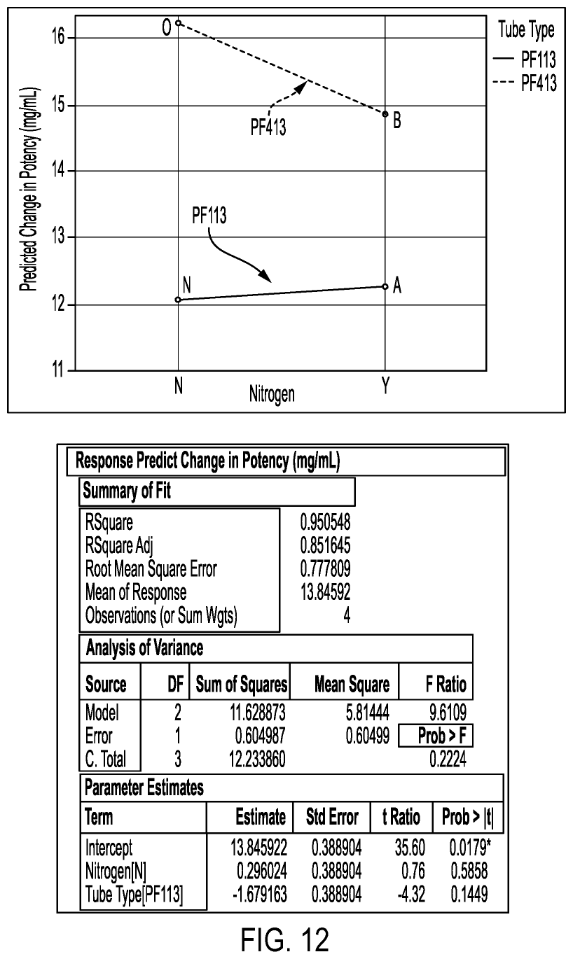

To compare the effect of using a nitrogen blanketed environment for filling, a sub-design of the overall experimental design (batches, A, B, N, and O) was considered. Those four batches all were produced without the use of BHA and/or BHT additives. FIG. 12 shows the predicted change versus Nitrogen (Y or N) and overlaid with the Tube Type. There is no indication of any statistically significant effect due to Nitrogen or the Tube Type. In addition, all of these formulations were identified as having very poor stability. Because of that, the use of solely filling under nitrogen does not appear to provide appropriate protection against degradation. While there is an apparent difference due to tube type, that difference was not statistically significant. The PF113 tube does, however, have better apparent stability (less change over time).

### Example 3

A transdermal buprenorphine solution was formulated as set forth in Table 12 below. The formulation contains 20 mg/mL buprenorphine (as hydrochloride) solubilized in dehydrated alcohol (ethanol). The solution also contains the excipient padimate O (50 mg/ml) as a skin penetration enhancer and BHA (0.39 mg/ml) and BHT (0.39 mg/ml). The formulation can be packaged using a unit-dose aluminum and polymer laminate tube having a 0.4 mL or 1.0 mL dose volume. This allows for a single dose of the formulation to be applied directly to a cat's skin.

TABLE 12
Transdermal formulation of buprenorphine

| Components | Quantity mg/mL | Function |
|---|---|---|
| Buprenorphine HCL | 20 | Active Substance |
| Padimate-O | 50 | Penetration Enhancer |
| BHA | 0.39 | Additive |
| BHT | 0.39 | Additive |
| Dehydrated Alcohol | Q.S. | Solvent |

### Example 4

To assess pharmacokinetics (PK), a transdermal buprenorphine solution (TBS) was formulated to contain 25 mg/mL buprenorphine (calculated as the free base), 5% w/v (50 mg/mL) padimate O as a permeation enhancer, and ethanol. To prepare the formulation, buprenorphine HCl (Spectrum Chemical Mfg. Corp., New Brunswick, NJ, USA) was dissolved in a small amount of ethanol (Sigma-Aldrich, St. Louis, MO, USA) and padimate O (Sigma-Aldrich) was added and mixed. The formulation was brought to volume with ethanol and aliquoted into 10 mL amber glass vials sealed with a rubber stopper and aluminum crimp-top until use. For the bioavailability phase of the study, the formulation was prepared in an identical manner, but to a final buprenorphine concentration of 20 mg/mL. The control article for the bioavailability portion of the study was buprenorphine hydrochloride injectable solution (Buprenex, Reckitt Benckiser Pharmaceuticals, Inc., Richmond, VA, USA).

For the first PK phase of the study, 12 adult domestic shorthaired cats (7 castrated males and 5 intact females) ranging in age from 1 to 4 years and weighing 2.35 to 6.35 kg were used. For the second bioavailability phase of the study, 12 adult domestic shorthaired male cats (6 castrated and 6 intact) ranging in age from 9 to 13 months and weighing 4.20 to 6.35 kg were used.

Animals were randomized to receive a single 10, 30, or 50 mg (n=4/group) dose of TBS. Randomization was blocked by bodyweight to maintain balance across dose groups. Animals were not fasted prior to treatment administration. TBS was administered topically to the unclipped skin on the dorsal cervical region (base of the skull) using the tip of a needleless syringe. The syringe tip was placed directly onto the skin at the application site and the entire dose volume was administered at a single location without moving the syringe. The volumes of TBS administered were 0.4, 1.2, and 2 mL in the 10, 30, or 50 mg dose group, respectively. The cats were gently restrained for 2 minutes post-dosing to prevent the cat from shaking or grooming while the solution dried. Blood samples (˜1 mL/sample) for plasma buprenorphine assay were collected from all cats prior to dosing and at 2, 4, 12, 24, 48, 72, and 168 hours post-dosing. Samples were collected by jugular venipuncture using a syringe and needle and immediately transferred into K₂EDTA tubes and placed on ice until plasma processing by centrifugation at 4° C. for 15 minutes at 1500 g. Plasma samples were stored frozen at −20° C. or below until analysis. Animal behavioral effects and mydriasis (0=no, 1=yes) were assessed, and rectal body temperatures measured prior to dose administration and at 2, 4, 12, 24, 48, 72,and 168 hours post-dosing. Behavior was scored on a 5-point scale as: 0—Normal; 1—Sedated (subdued and quiet; signs include sleeping, ventral tail curling, and purring; less responsive to human interaction); 2—Euphoric (exaggerated social and playful behavior; signs include meowing, rolling, kneading with forepaws, play-biting, and rubbing its head and body on cage); 3—Mildly Dysphoric (state of uneasiness and discord; signs include absent staring, hyper-responsiveness, swaying, and/or vocalization, and may be accompanied by increased locomotor activity; no overt signs of fear or disorientation, and no signs of aggression; may initially appear sedated, but then startle suddenly i.e., hyper-responsive); 4—Dysphoric (state of anxiety or agitation; signs include staring at objects that are not present, hyper-responsiveness, sudden movements, and/or vocalization, and may be accompanied by increased locomotor activity; cats are obviously disoriented or fearful, may become aggressive).

To assess bioavailability, animals were randomized to receive a single 20 mg dose of TBS (1 mL) or 0.05 mg IV buprenorphine (n=6/group). Randomization was blocked by bodyweight to maintain balance across dose groups. Animals were not fasted prior to treatment administration. Transdermal buprenorphine solution was administered topically in the same manner as in the first phase of the study i.e., on the dorsal cervical region. Injectable buprenorphine was administered IV as a bolus. To assure IV drug delivery, a temporary cephalic vein catheter was placed in each cat under sedation with 40 μg/kg IM dexmedetomidine hydrochloride (Dexdomitor, Zoetis Inc., Florham Park, NJ, USA). Sedation was reversed after catheter placement and prior to buprenorphine injection by administering 0.2 mg/kg IM atipamezole (Antisedan, Zoetis Inc.). Following IV administration of buprenorphine the catheters were flushed with sterile saline and removed from the animals. Blood samples (˜2 mL/samples) for plasma buprenorphine assay were collected from TBS treated cats prior to dosing and 1, 2, 4, 12, 24, 48, 96, 168, and 240 hours post-dosing and from IV buprenorphine cats prior to dosing and at 5 and 15 minutes, 1, 2, 4, 12 and 24 hours post-dosing. Samples were collected and stored as described in the first phase of the study.

For the first PK phase of the study, plasma samples were analyzed for buprenorphine concentration using a validated liquid chromatography tandem mass spectrometry (LC-MS/MS) method. A 100 μg/mL stock solution of buprenorphine HCl (Cerilliant®, Round Rock, TX, USA) was diluted into 50:50 methanol (Honeywell Burdick & Jackson, Morristown, NJ, USA): water to create working solution standards ranging from 0.500 to 1250 ng/ml and quality control (QC) working solutions of 3.00, 375, and 1000 ng/mL. Similarly, a 100 μg/mL stock solution of the internal standard (IS) buprenorphine-d4 (Cerilliant®, Round Rock, TX, USA) was diluted into acetonitrile to create a working IS solution of 10 ng/mL. Calibration and QC standards were then prepared by adding 10.0 μL of the appropriate working solution standards or QC working solutions to 50.0 μL of control blank feline plasma. Likewise 10.0 μL of 50:50 methanol: water was added to 50.0 μL of all study samples, blanks, and zero controls. Subsequently, 250 μL of the IS working solution was added to all calibration standard, QC, study, and zero control samples; and 250 μL of acetonitrile was added to all blanks. Samples were vortex-mixed for 2 minutes and then centrifuged for 10 minutes. Fifty (50.0) μL of the supernatant was transferred to a 96-well elution plate containing 250 μL of reverse osmosis water and vortex-mixed for 2 minutes. Water diluted samples were quantified using an API 5000™ triple quadrupole mass spectrometer equipped with TurbolonSpray™ interface (AB SCIEX, Framingham, MA, USA) with peak area integration conducted using Analyst Software v 1.5.1 (AB SCIEX) data acquisition system. HPLC separation was achieved using a Phenomenex Gemini C18 (50×3 mm, 5 um particle size) column (Phenomenex, Torrance, CA, USA) with he flow rate set at 0.700 mL/min and a column temperature of 30 μC. Mobile phase A consisted of 0.1% formic acid in water and mobile phase B consisted of 0.11% formic acid in acetonitrile. The mobile phase gradient started at 10% mobile phase B from 0.0 to 0.5 minutes, switched from 10% to 80% mobile phase B from 0.5 to 2.0 minutes, and switched back from 80% to 10% mobile phase B from 3.0 to 3.1 minutes. The injection volume was 5 μL and mass spectrometer detection was conducted using positive ionization mode and monitoring of the transitions 468.5 m/z→396.3 m/z for buprenorphine and 472.5 m/z→400.3 m/z for the IS buprenorphine-d4. Both analytes typically eluted from the column at 1.82 minutes. Standard curves were determined using linear regression with 1/x2 weighting using Excel (Version 11, Microsoft Corporation, Redmond, WA, USA), where x is the nominal sample concentration, and had typical squared correlation coefficient (R2) values of 0.9977-0.9993. All concentration calculations were based on the peak area ratios of buprenorpine to the IS. The calibration concentration range for buprenorphine was 0.100-250 ng/mL with a lower limit of quantification (LLOQ) of 0.100 ng/mL. The intra- and inter-assay precision (i.e., coefficient of variation) were ≤5.95% and the accuracy (i.e., relative error) ranged from 0.00% to 2.67%. A similar validated LC-MS/MS method was used for the second bioavailability phase of the study with modification. The calibration concentration range for buprenorphine was 0.200-100 ng/ml with a lower limit of quantification (LLOQ) of 0.200 ng/mL. The buprenorphine metabolite norbuprenorphine was demonstrated to not interfere with the quantification of plasma buprenorphine. The intra- and inter-assay precision (i.e., coefficient of variation) were ≤8.2% and the accuracy (i.e., relative error) ranged from −4.3% to 7.5%.

Plasma buprenorphine concentrations <LLOQ were excluded from the summary statistic calculations. PK parameters were calculated for each subject using noncompartmental PK analysis methods with Phoenix™ WinNonlin® Version 6.2 (Build 6.2.0.495, Pharsight®—A Certara Company, St. Louis, MO, USA). The linear trapezoidal rule was used for the area under the plasma concentration-time curve (AUC) calculations. The absolute bioavailability (F) of TBS was calculated as the ratio of the geometric means of the bodyweight dosage adjusted AUCs.

For the first phase of the study, the range of doses administered on bodyweight basis in the 10, 30, and 50 mg groups were 1.57-4.35, 4.72-13.03, and 7.87-21.73 mg/kg, respectively. Three plasma buprenorphine concentrations were considered outliers. Two concentrations from the 30 mg group excluded: a 12-hour sample was 54.6 ng/ml and a 48-hour sample was 72.6 ng/mL. A single concentration excluded from the 24-hour timepoint in the 10 mg treatment group that was 24.8 ng/mL. The reason for these outlier observations was not determined by study audit, but the measured concentrations were confirmed by re-assay. A sensitivity analysis was subsequently conducted on the plasma buprenorphine concentrations by removing these three observations and then re-calculating the plasma concentration summary statistics and the PK analysis. There was no difference with the outliers removed and therefore the samples remained excluded from the analysis below. For the first phase of the study, plasma buprenorphine reached peak mean concentrations between 2- and 4-hours post-dosing and all samples remained above the LLOQ through 168 hours post-dosing. The mean plasma buprenorphine concentrations in the 50 mg TBS group were occasionally marginally higher than, or the same as, those in the 30 mg dose group. The mean (range) Cₘₐₓ values were 10.5 (3.02-18.1), 18.6 (10.6-27.6), and 22.5 (19.5-29.0) ng/mL following 10, 30, and 50 mg TBS doses, respectively. The time of Cₘₐₓ occurrence (tₘₐₓ) ranged from 2 to 12 hours, except for a single value of 72 hours in the 10 mg dose group. The mean terminal half-lives (t_{1/2}) ranged from 78.3 to 91.2 hours. The mean percentages of AUC extrapolated ranged from 21.8% to 24.9% across dose groups. The mean (range) area under the curve from time 0 to infinity (AUC_{0-∞) were} 578 (218-967), 1590 (658-3310), and 2070 (1500-2710) hr·ng/mL following 10, 30, and 50 mg doses, respectively. There was 2.8- and 3.6-fold increase in AUC_{0∞} in the 30 and 50 mg dose group, respectively, compared to the 10 mg group. Transient sedation and euphoria were observed beginning within 2 hours post-dosing. Twenty-five to 50% of cats were sedated (behavioral score=1) for 24 hours in the 10 mg group. In the 30 and 50 mg groups, sedation was observed in 25 to 50% of cats for 48 hours, with no effects observed after 72 hours. Euphoria (behavioral score=2) was observed in 25 to 75% of cats in all dose groups through 24 hours with no euphoria observed beyond 72 hours. Neither mild dysphoria (behavioral score=3) nor dysphoria (behavioral score=4) was observed at any time in the study. Mean rectal body temperatures peaked at 12 hours post-dosing and appeared to be greater in the 30 and 50 mg TBS dose groups (38.9 and 39.1° C., respectively) than in the 10 mg dose group (38.5° C.). The mean temperatures remained from 0.6-0.9° C. greater than baseline (37.4-37.8° C.) through 168 hours post-dosing. Mydriasis was observed in 75% to 100% of cats in each dose group between 4- and 12-hours post-dosing. No mydriasis was observed in any cats in the 10 and 30 mg TBS dose groups from 48 hours post-doing onward. Mydriasis was observed in at least 50% of cats administered 50 mg TBS through 72 hours post-dosing. No mydriasis was observed in any cats beyond the 72 hours observation. For the second phase of the study, on a bodyweight basis, the mean (range) buprenorphine dosages following IV and TBS administration were 0.00972 (0.00787-0.0112) and 3.95 (3.33-4.76) mg/kg, respectively.

Plasma buprenorphine concentrations from the IV group rapidly decreased from a mean of 13.6 ng/ml at 5 minutes post-dosing to 0.231 ng/mL by 4 hours post-dosing and were <LLOQ beyond 4 hours. In contrast, the mean plasma buprenorphine concentrations from the TBS group peaked at 1 hour and gradually decreased; mean concentrations were 11.6, 7.11, 1.86, and 0.513 ng/ml at 1, 24, 96, and 240 hours post-dosing, respectively. The Cₘₐₓ and tₘₐₓ following TBS administration were 15.1 (4.82-25.6) ng/ml and 7.33 (1-24) hours, respectively and the initial concentration (C₀) following IV administration was 18.4 (14.2-27.5) ng/ml. The clearance (Cl) following IV administration was 16.7 (12.4-23.2) mL/min·kg. The t½ following IV and TBS administration were 0.82 (0.59-0.97) and 64.9 (39.1-85.7) hours, respectively. The percent AUC extrapolated was <20% for all subjects. The estimated absolute bioavailability (F) of TBS was 16.0% (90% CI: [11.8%-21.7%]).

Following topical application of a range of TBS doses in the present study, mean plasma buprenorphine concentrations exceeded 2.3 ng/ml at the 2-hour sample time for all three doses suggesting rapid onset of action. Mean terminal half-lives ranged from 78.3 to 91.2 hours across the 10, 30, and 50 mg TBS doses supporting extended duration. Mean buprenorphine concentrations exceeded 2.3 ng/ml through 168 hours at the 30 and 50 mg doses and were above 2.3 ng/ml for 72 hours at the 10 mg dose with the exception of the 48 hour sampling point. Further supporting a pharmacological effect, the plasma buprenorphine concentrations were temporally associated with behavioral and physiological effects consistent with opioid exposure including transient sedation, euphoria, mydriasis, and increased rectal temperature.

Mean plasma buprenorphine concentrations at sampling points from 2 to 72 hours ranged from 1.63-8.3, 4.61-17.1, and 7.90-22.3 ng/ml following 10, 30, and 50 mg TBS administration, respectively. Bioavailability was 16% (12.4-23.5%) which was near the estimated target and similar to estimates of buprenorphine bioavailability from patches in cats.

These results indicate that a single administration of TBS resulted in plasma buprenorphine concentrations likely to provide analgesia for multiple days at all examined doses although further studies of TBS across a range of doses are warranted to determine its analgesic efficacy. The product characteristics of TBS have the potential to overcome the limitations of other approved or compounded buprenorphine products used in cats including limited duration-of-action, the need for repeated administrations, dispensing controlled substances, end-of-dosing interval breakthrough pain, and offer the advantage of in-hospital, fear-free, stress-free administration, and prolonged duration-of-action. In addition, TBS can be readily included into a preventative pain management analgesic protocol at clinics.

### Example 5

A prospective, double-masked, placebo-controlled, multicentered phase 2 clinical study was conducted to select the transdermal buprenorphine solution (TBS) dosage for the control of postoperative pain in cats. The TBS was formulated into two strengths containing 16 and 20 mg/ml buprenorphine (calculated as free base), 5% w/v (50 mg/ml) padimate O, and ethanol. The negative control veterinary product was placebo transdermal solution containing 5% w/v (50 mg/ml) padimate O and ethanol. Transdermal solutions were packaged in 10 ml amber glass serum vials sealed with a rubber stopper and aluminum crimp-top until use.

One-hundred fifteen (115) cats were randomized to a single topical dose of placebo solution, low-TBS dosage (1.91-2.07 mg/kg) or high-TBS dosage (4.27-4.88 mg/kg) prior to surgical reproductive sterilization in conjunction with forelimb onychectomy. The dose administered was based on unit dosing for cats that fit into a body weight range. For cats allocated to the low-TBS dose, smaller cats (1.2-3 kg) received 4 mg and larger cats (>3-7.5 kg) received 10 mg. The 16 mg/mL TBS formulation was used in the low-TBS group resulting in dose volumes of 0.25 and 0.625 ml in the smaller and larger cats, respectively. For cats allocated to the high-TBS dose, smaller cats (1.2-3 kg) received 8 mg and larger cats (>3-7.5 kg) received 20 mg. The 20 mg/mL solution was used in the high-TBS group resulting in dose volumes of 0.4 and 1 ml in smaller and larger cats, respectively. Transdermal buprenorphine solution was administered 2-4 h prior to surgery for those allocated to the low-TBS dose and 1-2 h prior to surgery for those allocated to the high-TBS dose. In order to maintain masking, transdermal placebo solution was administered 1-2 h or 2-4 h prior to surgery according to randomization. Treatments were administered topically to the dorsal cervical region (base of the skull) using a needleless syringe by a single person assigned to treatment administration. The syringe tip was placed directly onto the skin at the application site by parting the hair, if necessary, and the entire dose volume was administered at a single location without moving the syringe.

Interactive pain assessments and physiological variables were quantified through 96 hours post-anesthetic recovery and rescue analgesia was administered any time that analgesia was considered inadequate. The estimated overall treatment success rates from generalized linear mixed effects model analysis were 0.10 (95% CI: [0.02-0.36]), 0.56 (95% CI: [0.25-0.83]), 0.71 (95% CI: [0.38-0.91]) in the placebo-, low-, and high-TBS dose groups, respectively. Success rates for both TBS treatment groups were superior to placebo. Adverse events were infrequent in all treatment groups although the postoperative body temperatures over the duration of the study were on average 0.31 (95% CI: [0.08-0.55]) and 0.30 (95% CI: [0.05-0.53])° C. higher in low- and high-TBS dose cats, respectively, compared to placebo. It was found that both the low- and high-TBS dosages were safe and effective.

### Example 6

A prospective, double masked, placebo-controlled, multicentered phase 3 clinical study was conducted to evaluate the safety and effectiveness of the transdermal buprenorphine solution (TBS) used in Example 5 for the control of post-operative pain in cats. A total of 228 cats from 12 US investigational sites met the enrollment criteria of which 107 placebo- and 112 TBS-treated cats were included into the per protocol efficacy analysis. The dose of TBS was 8 mg (0.4 ml) to cats 1.2 to 3 kilograms and 20 mg (1 ml) to cats >3 to 7.5 kilograms applied topically to the dorsal unclipped cervical skin 1-2 h prior to the undergoing elective surgical reproductive sterilization in conjunction with forelimb onychectomy. Interactive pain assessments and physiological variables were quantified through 96 h following recovery from anesthesia, and rescue analgesia was administered any time that pain control was scored inadequate. The treatment success rates were 0.40 (95% confidence interval [CI]: [0.28-0.53]) and 0.81 (95% CI: [0.70-0.89]) in the placebo and TBS groups, respectively, and the difference was significant (p<05). Adverse events occurred at a similar frequency and were not clinically meaningful in either treatment group. The post-operative body temperatures over the duration of the study were on average 0.35 (95% CI: [0.20-0.50])° C. higher than baseline in TBS-treated cats and were not clinically meaningful, an observation typical of opioids in cats. These results serve as substantial evidence that TBS is safe and effective for the control of orthopedic and soft tissue post-operative pain in cats when a single topical dose is applied 1-2 h prior to surgery.

### Example 7

Transdermal buprenorphine solution (TBS) was administered as a unit dose of 8 mg to cats weighing 1.2-3 kg and 20 mg to cats weighing to >3-7.5 kg, which is equivalent to a dosage on a bodyweight basis of 2.7-6.7 mg/kg. In this safety study, the 1× dose was defined as 6.7 mg/kg. Thirty-two cats (16 males and 16 females) were randomly allocated to placebo, 1, 2, and 3× TBS administered topically to the dorsal cervical skin every 4 days for 3 doses. Clinical observations, behavioral scores, mydriasis score (yes/no), and physiological variables were assessed or measured prior to each dose administration (0 h) and at 1, 2, 4, 8, 12, 24, 36, 48, and 72 h following each treatment and prior to euthanasia on Day 12 or 13. Blood samples for clinical pathology were collected on Days—1, 4, 8, and prior to euthanasia. There was little evidence of respiratory, cardiovascular, or gastrointestinal effects. Respiratory rates were above the reference range in all groups and lower by 10 breaths/min in the 3× group during the third dosing interval compared to placebo. There were no differences in heart rates. Constipation was transiently observed in approximately equal numbers in placebo- and TBS-treated cats. Behavioral scores showed sedation or euphoria was transient in the first dosing interval but became more prolonged with each dosing interval. Mydriasis was prolonged in the first dosing interval and diminished by the third dosing interval consistent with accommodation. Mean body temperatures in TBS-treated cats were up to 0.6° C. (1.8° F.) greater than placebo-treated cats. There were no clinically relevant changes to serum chemistry, hematology, or urinalysis outcomes nor gross or microscopic observations attributable to TBS. These data demonstrate that TBS is safe and well-tolerated when administered to 16-week-old cats at multiples of the approved dose and duration and supports clinical safety in the event of delayed buprenorphine metabolism, medication errors, or alterations in the dosing regimen.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

While reference has been made herein to certain examples, aspects, embodiments or the like, it is within the scope of this disclosure to combine the various elements of such examples, aspects, embodiments or the like with one another.

1. (canceled)

2. A method of treating pain in an animal in need thereof comprising transdermally administering a liquid pharmaceutical composition comprising a therapeutically effective amount of buprenorphine or salt thereof to the animal.

3. The method of claim 2, wherein the liquid pharmaceutical composition comprises a formulation comprising buprenorphine or salt thereof, and at least one additive selected from the group comprising butylated hydroxyanisole (BHA), butylated hydroxytoluene (BHT) and combinations thereof.

4. The method of claim 2, wherein the concentration of buprenorphine or salt thereof is about 5 mg/mL or more, about 10 mg/mL or more, about 15 mg/mL or more, about 20 mg/mL or more, about 25 mg/mL or more, about 30 mg/mL or more, about 35 mg/mL or more, or about 40 mg/mL or more.

5. (canceled)

6. The method of claim 2, wherein the buprenorphine or salt thereof comprises buprenorphine hydrochloride.

7. The method of claim 2, wherein the formulation further comprises a penetration enhancer.

8. (canceled)

9. (canceled)

10. The method of claim 7, wherein the penetration enhancer comprises padimate O.

11. The method of claim 2, wherein the formulation further comprises a solvent.

12. (canceled)

13. (canceled)

14. The method of claim 11, wherein the solvent comprises ethanol.

15. The method of claim 2, wherein the formulation is stable such that the concentration of buprenorphine degradation products in the formulation is less than that of an otherwise identical formulation not containing BHA and/or BHT after storage for up to 3 months, 6 months, 9 months, 12 months or 24 months at 25° C.

16. (canceled)

17. (canceled)

18. (canceled)

19. (canceled)

20. The method of any claim 15, wherein the buprenorphine degradation products comprise a polymer of buprenorphine.

21. The method of claim 2, wherein the buprenorphine degradation products comprise a dimer of buprenorphine and/or a positional isomer thereof.

22. The method of claim 2, wherein the concentration of the at least one additive is about 0.1 mg/mL or more, about 0.2 mg/mL or more, about 0.3 mg/mL or more, about 0.4 mg/mL or more, about 0.5 mg/mL or more, about 0.6 mg/mL or more, about 0.8 mg/mL or more, about 0.9 mg/mL or more, about 1.0 mg/mL or more, about 1.1 mg/mL or more, about 1.2 mg/mL or more, about 1.3 mg/mL or more, about 1.4 mg/mL or more, or about 1.5 mg/mL or more.

23. (canceled)

24. The method of claim 2, wherein the formulation further comprises BHA.

25. The method of claim 2, wherein the formulation further comprises BHT.

26. The method of claim 2, wherein the formulation further comprises BHA and BHT or the antioxidants in the formulation consist or consist essentially (i.e., constituting >90 wt. % or even 95 wt. % of the total amount of antioxidant) of BHA and BHT.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The method of claim 2, wherein the composition is administered at approximately the same temperature at which the composition is stored prior to administration.

40. The method of claim 2, wherein the composition is not heated prior to administration.

41. A formulation comprising buprenorphine or salt thereof, and at least one additive selected from the group comprising butylated hydroxyanisole (BHA), butylated hydroxytoluene (BHT) and combinations thereof.

42. The formulation of claim 41, wherein the buprenorphine or salt thereof comprises buprenorphine hydrochloride.

43. The formulation of claim 41, wherein the formulation further comprises a penetration enhancer.

44. The formulation of claim 41, wherein the formulation further comprises a solvent.

45. The formulation of claim 41, wherein the formulation is stable such that the concentration of buprenorphine degradation products in the formulation is less than that of an otherwise identical formulation not containing BHA and/or BHT after storage for up to 3 months, 6 months, 9 months, 12 months or 24 months at 25° C.

46. The formulation of claim 41, wherein the formulation comprises BHA.

47. The formulation of claim 41, wherein the formulation comprises BHT.

48. The formulation of claim 41, wherein the formulation comprises BHA and BHT or the antioxidants in the formulation consist or consist essentially (i.e., constituting >90 wt. % or even 95 wt. % of the total amount of antioxidant) of BHA and BHT.
